(19) United States
(12) Patent Application Publication
(10) Pub. No.: US 2022/0315887 A1
(43) Pub. Date: Oct. 6, 2022

(54) METHODS OF IMPROVING PROTEIN PRODUCTIVITY IN FED-BATCH CELL CULTURES

(71) Applicant: Bristol-Myers Squibb Company, Princeton, NJ (US)

(72) Inventors: Jianlin XU, Littleton, MA (US); Qin HE, Devens, MA (US); Mengmeng XU, Devens, MA (US); Matthew Stephen REHMANN, Devens, MA (US); Charles Anthony HILL, Groton, MA (US); Christopher Louis OLIVEIRA, Devens, MA (US); Michael Christopher BORYS, Gronton, MA (US); Zhengjian LI, Sudbury, MA (US); Shun ZHENG, Devens, MA (US)

(73) Assignee: Bristol-Myers Squibb Company, Princeton, NJ (US)

(21) Appl. No.: 17/632,211

(22) PCT Filed: Jul. 30, 2020
(86) PCT No.: PCT/US2020/044134

§ 371 Date: Feb. 1, 2022

Related U.S. Application Data
(60) Provisional application No. 62/989,560, filed on Mar. 13, 2020.
(60) Provisional application No. 62/881,668, filed on Aug. 1, 2019.

(51) Int. Cl. C12N 5/00; C12N 5/071; C12P 21/02; C12P 21/00

(52) U.S. Cl. CPC C12N 5/0018; C12N 5/0602; C12P 21/02; C12P 21/005; C12N 2511/00

(57) ABSTRACT

In certain embodiments, this disclosure provides a method of increasing production of a recombinant polypeptide of interest, comprising: a) seeding mammalian cells in a fed-batch production bioreactor at a viable cell density of at least 5106 viable cells/ml; and b) culturing the cells under optimized culture conditions to produce the recombinant polypeptide of interest at high titer.

### CROSS-REFERENCE TO RELATED APPLICATIONS

This application claims priority to U.S. Provisional Application Ser. No. 62/881,668, filed Aug. 1, 2019 and U.S. Provisional Application Ser. No. 62/989,560, filed Mar. 13, 2020, the entire contents of which is incorporated herein by reference.

### FIELD OF THE INVENTION

The present invention generally relates to methods of increasing production of a recombinant polypeptide of interest in fed-batch mammalian cell cultures.

### BACKGROUND OF THE INVENTION

Proteins and polypeptides have become increasingly important as therapeutic agents. In most cases, therapeutic proteins and polypeptides are produced in cell culture, from cells that have been engineered and/or selected to produce unusually high levels of the polypeptide of interest. Control and optimization of cell culture conditions is critically important for successful commercial production of proteins and polypeptides.

Perfusion cell culture can achieve much higher viable cell densities than conventional fed-batch cell culture systems. Perfusion cell culture provides a continuous supply of fresh media in the culture system, while removing waste products, which provides a rich environment for the cells to grow. However, perfusion cell culture becomes expensive when used in large-scale culture systems (e.g., greater than 200-L bioreactor) because of large quantities of cell culture media consumed. Also, perfusion cell culture can have complications from the cell retention system which prevents the cells from being removed from the cell culture system, especially for a large scale manufacturing. Therefore, the biopharmaceutical industry primarily uses fed-batch, rather than perfusion, to produce stable monoclonal antibodies (mAbs) from Chinese Hamster Ovary (CHO) cells.

Many proteins and polypeptides produced in cell culture are made in a fed-batch process, in which cells are cultured for a period of time, and then the culture is terminated and the produced protein or polypeptide is isolated. The ultimate amount and quality of protein or polypeptide produced can be dramatically affected by the N−1 seed culture and the seed-density at N production.

While efforts have been made to improve production of proteins and polypeptides in fed-batch culture processes, there remains a need for additional improvements.

### BRIEF SUMMARY OF THE INVENTION

In certain embodiments, this disclosure provides a method of increasing production of a recombinant polypeptide of interest, comprising: a) seeding mammalian cells in a fed-batch production bioreactor at a viable cell density of at least 5×10⁶ viable cells/ml; and b) culturing the cells under optimized culture conditions to produce the recombinant polypeptide of interest at high titer.

In certain aspects, the production stage bioreactor is a fed-batch bioreactor. In certain aspects, the seeding viable cell density in an N production stage is at least 10×10⁶, at least 15×10⁶, at least 20×10⁶, at least 25×10⁶, or at least 30×10⁶ viable cells per mL.

In some aspects, the titer of the recombinant polypeptide is at least 6 g/L, at least 8 g/L, at least 10 g/L, at least 15 g/L, at least 20 g/L, at least 25 g/L or at least 30 g/L on day 8, day 9, day 10 or later in N production stage (e.g., in a longer duration).

In some aspects, the cells at N production stage are cultured in a rebalanced basal medium. In other aspects, the cells at N production stage are cultured in an enriched basal medium. In some aspects, the cells at N production stage are fed with a rebalanced feed medium.

In some aspects, the feed is started at day 0, day 1, day 2, day 3, day 4 or day 5. In some embodiment, the daily feed percentage is at least 3%, at least 3.5%, at least 4%, at least 4.5% or at least 5% of initial culture volume. In some aspects, the daily feed percentage is kept the same. In other aspects, the daily feed percentage is varied, decreased or increased.

Optionally, the cell viability in N production stage is at least 50%, 60%, 70%, at least 75%, at least 80%, at least 85% or at least 90% over the whole production culture period. In some embodiments, the cell viability in N production stage is at least 70% over the whole production culture period.

In some aspects, the cells are seeded from an N−1 stage perfusion cell culture. In other aspects, the cells are seeded from an N−1 stage non-perfusion cell culture. In some aspects, the cells from N−1 stage are directly diluted and inoculated into N production stage. In other aspects, the cells from N−1 stage are concentrated before seeding into N production stage. For example, the cells from N−1 stage are concentrated by centrifugation or gravity settle down before seeding into N production stage.

In some aspects, the bioreactor is at least 50-L, at least 500-L, at least 1,000-L, at least 5,000-L, or at least 10,000-L scale.

In some aspects, the mammalian cells are selected from the group consisting of CHO, VERO, BHK, HEK, HeLa, COS, MDCK and hybridoma cells. In some embodiments, the mammalian cells are CHO cells.

In some aspects, the recombinant polypeptide of interest is an antibody or antigen-binding fragment. For example, the antibody or antigen-binding fragment binds an antigen selected from the group consisting of PD-1, PD-L1, CTLA-4, LAG-3, TIGIT, GITR, CXCR4, CD73, HER2, VEGF, CD20, CD40, CD11a, tissue factor (TF), PSCA, IL-8, EGFR, HER3, and HER4.

In some aspects, the cells are cultured at a single constant temperature over the whole production culture period. In other aspects, the cells are cultured at a shifted (e.g., decreased or increased) temperature over some of the culture period.

In some aspects, the method further comprises the step of isolating the polypeptide of interest from the production culture system.

### BRIEF DESCRIPTION OF THE DRAWINGS

FIGS. 1A-1 to 1A-4, 1B-1 to 1B-4, and 1C-1 to 1C-4 describe the effects of inoculation density, basal enrichment, feed start day and feed percentage on product titer by SAS JMP data analysis in high-throughput screening cell cultures run using 96 50-mL TubeSpin bioreactors with 32 conditions for each cell line (n=1). FIG. 1A shows cell line A data for mAb1 production. FIG. 1A-1 shows that mAb1 titer was significantly increased by increasing inoculation cell densities from 0.5, 3 and 6×10⁶ cells/mL (P<0.0001). FIG. 1A-2 shows that there was a significant interaction between inoculation cell density and basal medium enrichment on mAb1 production titer (P<0.0001). The more enriched media resulted in higher titer for higher inoculation cell density. FIG. 1A-3 shows that there was a significant interaction between inoculation cell density and feed start day on mAb1 production titer (P<0.0001). The earlier feeding day resulted in higher titer for higher inoculation cell density. FIG. 1A-4 shows that there was a significant interaction between inoculation cell density and feed percentage on mAb1 production titer (P=0.0001). The higher feed percentage resulted in higher titer for higher inoculation cell density.

FIG. 1B shows cell line B data for mAb2 production. FIG. 1B-1 shows that mAb2 titer was significantly increased by increasing inoculation cell densities from 0.5, 3 and 6×10⁶ cells/mL (P<0.0001). FIG. 1B-2 shows that there was a significant interaction between inoculation cell density and basal medium enrichment on mAb2 production titer (P=0.0002). The more enriched media resulted in higher titer for higher inoculation cell density. FIG. 1B-3 shows that there was a significant interaction between inoculation cell density and feed start day on mAb2 production titer (P=0.0362). The earlier feeding day resulted in higher titer for higher inoculation cell density. FIG. 1B-4 shows that there was a significant interaction among inoculation cell density, basal medium enrichment, and feed start day on mAb2 production titer (P=0.0318). The earlier feed start day and higher feed percentage resulted in higher titer for higher inoculation cell density. FIG. 1C shows cell line C data for mAb3 production. FIG. 1C-1 shows that mAb3 titer was significantly increased by increasing inoculation cell densities from 0.5, 3 and 6×10⁶ cells/mL (P<0.0001). FIG. 1C-2 shows that there was a significant interaction between inoculation cell density and basal medium enrichment on mAb3 production titer (P<0.0001). The more enriched media resulted in higher titer for higher inoculation cell density. FIG. 1C-3 shows that there was a significant interaction between inoculation cell density and feed start day on mAb3 production titer (P=0.0031). The earlier feeding day resulted in higher titer for higher inoculation cell density. FIG. 1C-4 shows that there was a significant interaction between inoculation cell density and feed percentage on mAb3 production titer (P=0.0078). The higher feed percentage resulted in higher titer for higher inoculation cell density.

FIGS. 2A, 2B and 2C show the comparison of Process B in 1000-L bioreactors and Process C in 5-L bioreactors for mAb1 production. The media and process changes are shown in Table 2 and Table 3, respectively. FIG. 2A shows viable cell density (VCD) profiles and FIG. 2B shows cell viability profiles for the enriched N−1 seed at 200-L scale for Process B and the perfusion N−1 seed at lab scales for Process C. The N−1 seed culture for Process C reached much higher final VCD because of perfusion, while the cell viability profiles were similar. FIG. 2C shows VCD profiles, FIG. 2D shows viability profiles and FIG. 2E shows titer profiles at 5-L and 1000-L scales using the same 200-L N−1 batch seed for Process B, and at 5-L scale using the perfusion seed for Process C. The VCD in fed-batch production for Process C was much higher than the VCD in the fed-batch production for Process B. Although the viability for Process C was slightly lower than Process B, the viability for both processes remained high (above 95%) until day 10. Process C achieved approximately double the titer of Process B for the entire culture duration. The increased titer and volumetric productivity in Process C were mainly attributed to significantly higher VCD in the production stage, since the cell specific productivities were comparable between Process B and Process C (FIG. 2E and Table 4).

FIGS. 3A and 3B show the effects of inoculation cell density or seeding density (SD) and media change on cell culture performance for mAb2 production in 5-L fed-batch bioreactors. The media change is shown in Table 5. The experimental design is shown in Table 6. FIG. 3A shows VCD profiles for different conditions (Table 6). The higher inoculation density gave a higher peak VCD with the same basal and feed media. For the same inoculation density, Process C media condition had higher peak VCD than Process B media condition. FIG. 3B shows titer profiles for different conditions (Table 6). Increasing inoculation density did not improve final titer significantly with Process B media (Solid lines), while increasing inoculation density improved final titer significantly with Process C media (dashed lines). The change in media from control (solid triangle line) to Process C media only (dashed triangle line) slightly improved titer for the control inoculation density at 3×10⁶ cells/mL. Only the conditions (dashed square line and dashed circle line) with both increasing inoculation cell density and Process C media resulted in significant titer improvement.

FIGS. 4A, 4B, 4C, 4D and 4E show the large scale bioreactor performance of N−1 seed and fed-batch production bioreactors, and the in-process quality attributes for mAb2 Process B and Process C. The media and process parameter changes made from mAb2 Process B to Process C are summarized in Table 5 and Table 7, respectively. For N−1 cultures, the perfusion seed culture achieved a much higher final VCD of about 100×10⁶ cells/mL for Process C when compared with the enriched batch N−1 culture which achieved a final VCD of about 16×10⁶ cells/mL for Process B (FIG. 4A). Due to the high final VCD for the perfusion N−1 seed, the viability dropped to about 95% on day 6, while the enriched batch N−1 seed for Process B maintained cell viability above 99% for the entire 4-day duration (FIG. 4B). In the production bioreactor, the VCD for Process C with 16×10⁶ cells/mL SD was not only much higher in the beginning of culture than the VCD for Process B, but Process C also maintained higher VCD through the entire 14-day duration (FIG. 4C). Due to lower viability at the end of the perfusion N−1 step for Process C (FIG. 4B), the viability for Process C was slightly lower than in Process B at the beginning of the fed-batch production, but the Process C viability increased and trended similarly to Process B from day 2 on (FIG. 4D). There was a viability dip in the middle of the run for Process B, which was not seen for Process C (FIG. 4D). Importantly, the titer for Process C was approximately doubled compared to the titer of Process B throughout the entire 14-day duration (FIG. 4E and Table 8), while in-process quality attributes, e.g. charge variant species, N glycans, and SEC impurities, were similar between Process B and C (FIG. 4F and Table 9). The doubled titer and volumetric productivity for mAb2 Process C compared with Process B can be attributed to both higher VCD and higher cell specific productivity (Table 8).

FIGS. 5A, 5B and 5C show the comparison of mAb4 cell culture performance in large scale bioreactors for Process B and Process C. The media and process parameters for Process B and Process C are shown in Table 10 and Table 11, respectively. FIG. 5A shows VCD profiles for Process B and Process C, described in Table 11. The VCD profile including peak VCD for Process C in 500-L bioreactors were almost doubled over Process B in 1000-L bioreactors (FIG. 5A). The cell viability for Process C was slightly lower than Process B in the end of cell culture (FIG. 5B), titer for Process C was doubled (FIG. 5C), while in-process quality attributes, e.g., charge variant species, N glycans and SEC impurities, were similar between Process B and C (FIG. 5D and Table 13). The two-fold increases in titer and volumetric productivity for Process C were mainly attributed to the much higher VCD compared to Process B, while cell specific productivity was similar between Process B and Process C (FIG. 5D and Table 12).

FIGS. 6A, 6B, 6C, 6D, 6E and 6F show the comparison of mAb5 cell culture performance in 1000-L bioreactors for Process A and in 5-L and 500-L bioreactors for Process C. The media and process parameters for Process A and Process C are shown in Table 14 and Table 15, respectively. The N−1 perfusion seed culture reached a final VCD of more than 100×10⁶ cells/mL for Process C, which was more than 10-fold higher than the batch N−1 seed with a final VCD of about 8×10⁶ cells/mL for Process A (FIG. 6A). The final viability Process C was slightly lower than the batch seed (FIG. 6B). Due to the high SD at 15×10⁶ cells/mL, the VCD profile for Process C was significantly higher than Process A with a SD of only 1.5×10⁶ cells/mL, while the 500-L achieved a slightly higher VCD profile than 5-Ls for Process C (FIG. 6C). The cell viability profiles were similar for both Process A at 1000-L and Process C at 500-L for the entire duration, while lower cell viability was observed at 5-L scale (FIG. 6D). The titer was more than tripled from Process A to Process C (FIG. 6E and Table 16), while quality attributes were comparable from Process A to Process C (FIG. 6F).

### DETAILED DESCRIPTION OF THE INVENTION

### Definitions

The indefinite articles “a” or “an” should be understood to refer to “one or more” of any recited or enumerated component.

The term “about” as used herein to a value or composition that is within an acceptable error range for the particular value or composition as determined by one of ordinary skill in the art, which will depend in part on how the value or composition is measured or determined, i.e., the limitations of the measurement system. For example, “about” can mean within 1 or more than 1 standard deviation per the practice in the art. Alternatively, “about” can mean a range of up to 20%. Furthermore, particularly with respect to biological systems or processes, the terms can mean up to an order of magnitude or up to 5-fold of a value. When particular values or compositions are provided in the application and claims, unless otherwise stated, the meaning of “about” should be assumed to be within an acceptable error range for that particular value or composition.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone). The use of the alternative (e.g., “or”) should be understood to mean either one, both, or any combination thereof of the alternatives.

As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a D-amino acid; in some embodiments, an amino acid is an L-amino acid. Amino acids, including carboxy- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc. In some embodiments, amino acids of the present invention may be provided in or used to supplement medium for cell cultures. In some embodiments, amino acids provided in or used to supplement cell culture medium may be provided as salts or in hydrate form.

The terms “polynucleotide” and “nucleotide” as used herein are intended to encompass a singular nucleic acid as well as plural nucleic acids, and refers to an isolated nucleic acid molecule or construct, e.g., messenger RNA (mRNA), complementary DNA (cDNA), or plasmid DNA (pDNA). In certain aspects, a polynucleotide comprises a conventional phosphodiester bond or a non-conventional bond (e.g., an amide bond, such as found in peptide nucleic acids (PNA)).

The term “polypeptide” as used herein refers to a molecule composed of monomers (amino acids) linearly linked by amide bonds (also known as peptide bonds). The term “polypeptide” refers to any chain or chains of two or more amino acids, and does not refer to a specific length of the product. As used herein the term “protein” is intended to encompass a molecule comprised of one or more polypeptides, which can in some instances be associated by bonds other than amide bonds. On the other hand, a protein can also be a single polypeptide chain. In this latter instance the single polypeptide chain can in some instances comprise two or more polypeptide subunits fused together to form a protein. The terms “polypeptide” and “protein” also refer to the products of post-expression modifications, including without limitation glycosylation, acetylation, phosphorylation, amidation, derivatization by known protecting/blocking groups, proteolytic cleavage, or modification by non-naturally occurring amino acids. A polypeptide or protein can be derived from a natural biological source or produced by recombinant technology, but is not necessarily translated from a designated nucleic acid sequence. It can be generated in any manner, including by chemical synthesis.

The term “polypeptide of interest” as used herein is used in its broadest sense to include any protein (either natural or recombinant), present in a mixture, for which purification is desired. Such polypeptides of interest include, without limitation, enzymes, hormones, growth factors, cytokines, immunoglobulins (e.g., antibodies), and/or any fusion proteins.

The terms “recombinantly expressed polypeptide” and “recombinant polypeptide” as used herein refer to a polypeptide expressed from a mammalian host cell that has been genetically engineered to express that polypeptide. The recombinantly expressed polypeptide can be identical or similar to polypeptides that are normally expressed in the mammalian host cell. The recombinantly expressed polypeptide can also be foreign to the host cell, i.e. heterologous to peptides normally expressed in the mammalian host cell. Alternatively, the recombinantly expressed polypeptide can be chimeric in that portions of the polypeptide contain amino acid sequences that are identical or similar to polypeptides normally expressed in the mammalian host cell, while other portions are foreign to the host cell.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies with two different heavy/light chain pairs and two different binding site, monospecific antibodies, monovalent antibodies, chimeric antibodies, humanized antibodies, human antibodies, fusion proteins comprising an antigen determination portion of an antibody, and any other modified immunoglobulin molecule comprising an antigen recognition site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antigen-binding portion” of an antibody, or an “antigen-binding fragment”, as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding fragment”, e.g., (i) a Fab fragment (fragment from papain cleavage) or a similar monovalent fragment consisting of the VL, VH, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; (vi) an isolated complementarity determining region (CDR) and (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “batch” or “batch culture” as used herein refers to a method of culturing cells in which all the components that will ultimately be used in culturing the cells, including the basal medium (see definition of “basal medium” below) as well as the cells themselves, are provided at the beginning of the culturing process. A batch culture is typically stopped at some point and the cells and/or components in the medium are harvested and optionally purified.

The term “basal medium” as used herein refers to a solution containing nutrients which nourish growing mammalian cells. Typically, these solutions provide essential and non-essential amino acids, vitamins, energy sources, lipids, and trace elements required by the cell for minimal growth and/or survival. The solution may also contain components that enhance growth and/or survival above the minimal rate, including hormones and growth factors. The solution is preferably formulated to a pH and salt concentration optimal for cell survival and proliferation. Various components may be added to a basal medium to benefit cell growth. The medium may also be a “chemically-defined medium” that does not contain serum, hydrolysates or components of unknown composition. Defined media are free of animal-derived components and all components have a known chemical structure.

The terms “rebalanced basal medium” as used herein refers to a modified basal medium with increased concentrations of some components and/or with decreased concentrations of some other components such that it provides for better cell growth and protein production.

The term “enriched basal medium” as used herein refers to a modified basal medium with the addition of concentrated dry powder media such that it has higher nutrient concentrations.

The term “fed-batch” or “fed-batch culture” means the incremental or continuous addition of a feed medium to an initial cell culture without substantial or significant removal of the initial basal medium from the cell culture. In some instances, the feed medium is the same as the initial basal medium. In other instances, the second liquid culture medium is a concentrated form of the basal medium and/or is added as a dry powder.

The term “rebalanced feed medium” as used herein refers to a modified fed medium with increased concentrations of some components and/or with decreased concentrations of some other components such that it provides for better cell growth and protein production.

The term “viable cell density” as used herein refers to the number of viable (living) cells present in a given volume of medium.

The term “cell viability” as used herein refers to the ability of cells in culture to survive under a given set of culture conditions or experimental variations. The term as used herein also refers to the portion of cells which are alive at a particular time in relation to the total number of cells, living and dead, in the culture at that time.

The terms “seeding”, “seeded”, “inoculation”, “inoculating” and “inoculated” as used herein refer to the process of providing a cell culture to a bioreactor or another vessel. The cells may have been propagated previously in another bioreactor or vessel. Alternatively, the cells may have been frozen and thawed immediately prior to providing them to the bioreactor or vessel. The term refers to any number of cells, including a single cell.

The terms “culture”, “cell culture” and “mammalian cell culture” as used herein refer to a mammalian cell population that is suspended in a medium under conditions suitable to survival and/or growth of the cell population. As will be clear to those of ordinary skill in the art, these terms as used herein may refer to the combination comprising the mammalian cell population and the medium in which the population is suspended.

The term “culturing” or “cell culturing” means the maintenance or growth of a mammalian cell in a liquid culture medium under a controlled set of physical conditions.

The term “N−1 stage” as used herein refers to the last seed expansion stage right before production inoculation. The N−1 stage is the final cell growth step before seeding the production bioreactor for polypeptide production. The terms “N−2 stage” and “N−3 stage” as used herein refers to the period of time during cell growth and expansion and, typically, before inoculation of N production stage. The N−3 stage is the cell growth stage used to increase viable cell density to be used in the N−2 stage. The N−2 stage is the cell growth stage used to increase viable cell density to be used in the N−1 stage.

The term “production stage” or “N production stage” of the cell culture refers to last stage of cell culture. During the production stage, cells will grow first and then followed with polypeptide production. The production stage is commonly referred to as “N” or last stage of cell culture manufacturing.

The term “bioreactor” as used herein refers to any vessel used for the growth of a mammalian cell culture. The bioreactor can be of any size so long as it is useful for the culturing of mammalian cells. Typically, the bioreactor will be at least 1 liter and may be 10, 100, 250, 500, 1000, 2500, 5000, 8000, 10,000, 12,000, 15,000, 20,000 liters or more, or any volume in between. The internal conditions of the bioreactor, including, but not limited to pH and temperature, are typically controlled during the culturing period. The bioreactor can be composed of any material that is suitable for holding mammalian cell cultures suspended in media under the culture conditions of the present invention, including glass, plastic or metal. The term “production bioreactor” as used herein refers to the final bioreactor used in the production of the polypeptide or protein of interest. The volume of the large-scale cell culture production bioreactor is typically at least 500 liters and may be 1,000, 2,500, 5,000, 8,000, 10,000, 12,000, 15,000, 20,000 liters or more, or any volume in between. One of ordinary skill in the art will be aware of and will be able to choose suitable bioreactors for use in practicing the present invention.

The term “perfusion” or “perfusion process” as used herein refers to a method of culturing cells in which equivalent volumes of media (containing nutritional supplements) are simultaneously added and removed from the bioreactor while the cells are retained in the reactor. A volume of cells and media corresponding to the supplement media is typically removed on a continuous or semi-continuous basis and is optionally purified. Typically, a cell culture process involving a perfusion process is referred to as “perfusion culture.” In some embodiments, a fresh medium may be identical or similar to the base medium used in the cell culture process. In some embodiments, a fresh medium may be different than the base medium but contain the desired nutritional supplements. In some embodiments, a fresh medium is a chemically-defined medium.

The terms “purifying,” “separating,” “isolating,” or “recovering,” as used interchangeably herein, refer to at least partially purifying or isolating (e.g., at least or about 5%, e.g., at least or about 10%, 15%, 20%, 25%, 30%, 40%, 45%, 50%, 55%, 60%, 65%, 70%, 75%, 80%, 85%, 90%, or at least or about 95% pure by weight) a recombinant protein from one or more other components present in the cell culture medium (e.g., mammalian cells or culture medium proteins) or one or more other components (e.g., DNA, RNA, or other proteins) present in a mammalian cell lysate. Typically, the degree of purity of the protein of interest is increased by removing (completely or partially) at least one impurity from the composition.

The term “shake flask” is meant a vessel (e.g., a sterile vessel) that can hold a volume of liquid culture medium that has at least one gas permeable surface. For example, a shake flask can be a cell culture flask, such as a T-flask, an Erlenmeyer flask, or any art-recognized modified version thereof.

The term “titer” as used herein refers to the total amount of recombinantly expressed polypeptide or protein produced by a mammalian cell culture divided by a given amount of medium volume. Titer is typically expressed in units of grams of polypeptide or protein per liter of medium. The term “high titer” as used herein refers to the a concentration at least 6 g/L, at least 8 g/L, at least 10 g/L, at least 15 g/L, at least 20 g/L, at least 25 g/L, at least 30 g/L, at least 40 g/L or at least 50 g/L at the N production stage (for example, on day 8, day 9, day 10 or later in N production stage. Various aspects of the disclosure are described in further detail in the following subsections.

In certain embodiments, this disclosure provides a method of increasing production of a recombinant polypeptide of interest, comprising: a) seeding mammalian cells in a fed-batch production bioreactor at a viable cell density of at least 5×10⁶ viable cells/ml; and b) culturing the cells under optimized culture conditions to produce the recombinant polypeptide of interest at high titer.

In certain aspects, the production stage bioreactor is a fed-batch bioreactor. In certain aspects, the seeding viable cell in an N production stage is at least 10×10⁶, at least 15×10⁶, at least 20×10⁶, at least 25×10⁶, or at least 30×10⁶ viable cells per mL. In some aspects, the titer of the recombinant polypeptide is at least 6 g/L, at least 8 g/L, at least 10 g/L, at least 15 g/L, at least 20 g/L, at least 25 g/L or at least 30 g/L on day 8, day 9, day 10 or later in N production stage.

In some aspects, the cells at N production stage are cultured in a rebalanced basal medium. In other aspects, the cells at N production stage are cultured in an enriched basal medium. In some aspects, the cells at N production stage are fed with a rebalanced feed medium.

In some aspects, the feed is started at day 0, day 1, day 2, day 3, day 4 or day 5. In some embodiment, the daily feed percentage is at least 3%, at least 3.5%, at least 4%, at least 4.5% or at least 5% of initial culture volume. In some aspects, the daily feed percentage is kept the same. In other aspects, the daily feed percentage is varied, decreased or increased.

Optionally, the cell viability in N production stage is at least 50%, 60%, 70%, at least 75%, at least 80%, at least 85% or least 90% over the whole production culture period. In some embodiments, the cell viability in N production stage is at least 70% over the whole production culture period.

In some aspects, the cells are seeded from an N−1 stage perfusion cell culture. In other aspects, the cells are seeded from an N−1 stage non-perfusion cell culture. In some aspects, the cells from N−1 stage are directly diluted and inoculated into N production stage. In other aspects, the cells from N−1 stage are concentrated before seeding into N production stage. For example, the cells from N−1 stage are concentrated by centrifugation or gravity settle down before seeding into N production stage.

In some aspects, the bioreactor is at least 50-L, at least 500-L, at least 1,000-L, at least 5,000-L, or at least 10,000-L scale.

In some aspects, the mammalian cells are selected from the group consisting of CHO, VERO, BHK, HEK, HeLa, COS, MDCK and hybridoma cells. In some embodiments, the mammalian cells are CHO cells.

In some aspects, the recombinant polypeptide of interest is an antibody or antigen-binding fragment. For example, the antibody or antigen-binding fragment binds an antigen selected from the group consisting of PD-1, PD-L1, CTLA-4, LAG-3, TIGIT, GITR, CXCR4, CD73 HER2, VEGF, CD20, CD40, CD11a, tissue factor (TF), PSCA, IL-8, EGFR, HER3, and HER4.

In some aspects, the cells are cultured at a single constant temperature over the whole production culture period. In other aspects, the cells are cultured at a shifted (e.g., decreased or increased) temperature over some of the culture period.

In some aspects, the method further comprises the step of isolating the polypeptide of interest from the production culture system.

### Host Cells

Any mammalian cell or cell type susceptible to cell culture, and to expression of polypeptides, may be utilized in accordance with the present invention. Non-limiting examples of mammalian cells that may be used in accordance with the present invention include BALB/c mouse myeloma line (NSO/1, ECACC No: 85110503); human retinoblasts (PER.C6 (CruCell, Leiden, The Netherlands)); monkey kidney CV1 line transformed by SV40 (COS-7, ATCC CRL 1651); human embryonic kidney line (293 or 293 cells subcloned for growth in suspension culture, Graham et al., J. Gen Virol., 36:59 (1977)); baby hamster kidney cells (BHK, ATCC CCL 10); Chinese hamster ovary cells ±DHFR (CHO, Urlaub and Chasin, Proc. Natl. Acad. Sci. USA, 77:4216 (1980)); mouse sertoli cells (TM4, Mather, Biol. Reprod., 23:243-251 (1980)); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCLS 1); TM cells (Mather et al., Annals N.Y. Acad. Sci., 383:44-68 (1982)); MRC 5 cells; FS4 cells; and a human hepatoma line (Hep G2). In one embodiment, the present invention is used in the culturing of and expression of polypeptides and proteins from CHO cell lines.

Additionally, any number of commercially and non-commercially available hybridoma cell lines that express polypeptides or proteins may be utilized in accordance with the present invention. One skilled in the art will appreciate that hybridoma cell lines might have different nutrition requirements and/or might require different culture conditions for optimal growth and polypeptide or protein expression, and will be able to modify conditions as needed.

As noted above, in many instances the cells will be selected or engineered to produce high levels of protein or polypeptide. Often, cells are genetically engineered to produce high levels of protein, for example by introduction of a gene encoding the protein or polypeptide of interest and/or by introduction of control elements that regulate expression of the gene (whether endogenous or introduced) encoding the polypeptide of interest.

Certain polypeptides may have detrimental effects on cell growth, cell viability or some other characteristic of the cells that ultimately limits production of the polypeptide or protein of interest in some way. Even amongst a population of cells of one particular type engineered to express a specific polypeptide, variability within the cellular population exists such that certain individual cells will grow better and/or produce more polypeptide of interest. In certain embodiments of the present invention, the cell line is empirically selected by the practitioner for robust growth under the particular conditions chosen for culturing the cells. In other embodiments, individual cells engineered to express a particular polypeptide are chosen for large-scale production based on cell growth, final cell density, percent cell viability, titer of the expressed polypeptide or any combination of these or any other conditions Deemed Important by the Practitioner.

### Fed-Batch Cell Culture Production

Typical procedures for producing a polypeptide of interest include perfusion or non-perfusion cultures for seed expansion and fed-batch culture production stage. After cells from seed cultures grow to a particular cell density under conditions conducive to cell growth and viability, the seed cultures are transferred to the next stage. Fed-batch culture procedures include a feed step or steps of supplementing the batch culture with nutrients and other components that are consumed during the growth of the cells. One of ordinary skill in the art will recognize that the present invention can be employed in any system in which cells are cultured including, but not limited to, batch, fed-batch and perfusion systems.

In certain preferred embodiments, the cells are grown in the fed-batch production stage at a high seeding viable cell density of at least 5×10⁶, at least 10×10⁶, at least 15×10⁶, at least 20×10⁶, at least 25×10⁶, or at least 30×10⁶ viable cells per mL. In certain preferred embodiments, the high-seed viable density cells in the fed-batch production stage are inoculated from N−1 perfusion culture. In certain embodiments, the high-seed viable density cells in the fed-batch production stage are inoculated from N−1 Non-perfusion culture.

The present invention provides balanced and enriched, chemically-defined basal media formulations that, when used in accordance with other culturing steps described herein, increase the titer of the recombinant polypeptide of interest in the production culture with high-seed density, relative to host cells cultured in non-enriched or non-balanced media. In certain embodiments, the cells are grown in a balanced basal medium. In other embodiments, the cells are grown in an enriched basal medium.

Feed optimization in the present invention also benefits the production of the recombinant polypeptide of interest in the N fed batch. In certain embodiments, the rebalanced feed medium is applied to feed on day 1, day 2, day 3 or day 4. In certain embodiments, wherein the feed percentage is at least 3% of initial culture volume, at least 4% of initial culture volume, or at least 5% of initial culture volume. In certain embodiments, the feed percentage is fixed. In certain embodiments, the feed percentage is varied over the whole production stage.

The temperature of the cell culture at the production stage will be selected based primarily on the range of temperatures at which the cell culture remains viable. In general, most mammalian cells grow well within a range of about 25° C. to 42° C. Preferably, mammalian cells grow well within the range of about 30° C. to 40° C. Those of ordinary skill in the art will be able to select appropriate temperature or temperatures in which to grow cells, depending on the needs of the cells and the production requirements of the practitioner. Optionally, the temperature is maintained at a single, constant temperature. Optionally, the temperature is maintained within a range of temperatures. For example, the temperature may be steadily increased or decreased. Alternatively, the temperature may be increased or decreased by discrete amounts at various times. In certain embodiments, the cells are grown at a higher temperature first, then a lower temperature later. In certain embodiments, the lower temperature is about 30° C., about 31° C., about 32° C. or about 33° C. In certain embodiments, the temperature shift occurs on day 1, day 2, day 3, day 4, day 5, day 6, day 7 or any other days during the cell culture after seeding. One of ordinary skill in the art will be able to determine whether a single or multiple temperatures should be used, and whether the temperature should be adjusted steadily or by discrete amounts.

In accordance with the present invention, the production bioreactor can be any volume that is appropriate for large-scale production of polypeptides or proteins. In a certain embodiment, the volume of the production bioreactor is at least 500 liters. In other embodiments, the volume of the production bioreactor is 1,000, 2,500, 5,000, 8,000, 10,000, 15,000, or 20,000 liters or more, or any volume in between. One of ordinary skill in the art will be aware of and will be able to choose a suitable bioreactor for use in practicing the present invention. The production bioreactor may be constructed of any material that is conducive to cell growth and viability that does not interfere with expression or stability of the produced polypeptide or protein.

Any of these media formulations disclosed in the present invention may optionally be supplemented as necessary with hormones and/or other growth factors, particular ions (such as sodium, chloride, calcium, magnesium, and phosphate), buffers, vitamins, nucleosides or nucleotides, trace elements (inorganic compounds usually present at very low final concentrations), amino acids, lipids, protein hydrolysates, or glucose or other energy source. In certain embodiments of the present invention, it may be beneficial to supplement the media with chemical inductants such as hexamethylene-bis (acetamide) (“HMBA”) and sodium butyrate (“NaB”). These optional supplements may be added at the beginning of the culture or may be added at a later point in order to replenish depleted nutrients or for another reason. One of ordinary skill in the art will be aware of any desirable or necessary supplements that may be included in the disclosed media formulations.

### Providing a Mammalian Cell Culture

Once a cell that expresses the polypeptide or protein of interest has been identified, the cell is propagated in culture by any of the variety of methods well-known to one of ordinary skill in the art. The cell expressing the polypeptide or protein of interest is typically propagated by growing it at a temperature and in a medium that is conducive to the survival, growth and viability of the cell. The initial culture volume can be of any size, but is often smaller than the culture volume of the production bioreactor used in the final production of the polypeptide or protein of interest, and frequently cells are passaged several times in bioreactors of increasing volume prior to seeding the production bioreactor. Once the cells have reached a specific viable cell density, the cells are grown in a bioreactor to further increase the number of viable cells. These bioreactors are referred to as N−1, N−2, N−3, and etc. “N” refers to the main production culture bioreactor, while the “N−1” means the bioreactor prior to the main production culture, and so forth.

The cell culture can be agitated or shaken to increase oxygenation of the medium and dispersion of nutrients to the cells. Alternatively or additionally, special sparging devices that are well known in the art can be used to increase and control oxygenation of the culture. In accordance with the present invention, one of ordinary skill in the art will understand that it can be beneficial to control or regulate certain internal conditions of the bioreactor, including but not limited to pH, temperature, oxygenation, etc.

Generally, cell cultures of N−1 may be grown to a desired density before seeding the next production bioreactor. It is preferred that most of the cells remain alive prior to seeding, although total or near total viability is not required. In one embodiment of the present invention, the cells may be removed from the supernatant, for example, by low-speed centrifugation. It may also be desirable to wash the removed cells with a medium before seeding the next bioreactor to remove any unwanted metabolic waste products or medium components. The medium may be the medium in which the cells were previously grown or it may be a different medium or a washing solution selected by the practitioner of the present invention.

The cells of N−1 may then be diluted to an appropriate density for seeding the production bioreactor. In a certain embodiment of the present invention, the cells are diluted into the same medium that will be used in the production bioreactor. Alternatively, the cells can be diluted into another medium or solution, depending on the needs and desires of the practitioner of the present invention or to accommodate particular requirements of the cells themselves, for example, if they are to be stored for a short period of time prior to seeding the production bioreactor.

The cells at the N−1 stage or the production stage may be grown for a greater or lesser amount of time, depending on the needs of the practitioner and the requirement of the cells themselves. In one embodiment, the cells are grown for a period of time sufficient to achieve a viable cell density that is a given percentage of the maximal viable cell density that the cells would eventually reach if allowed to grow undisturbed. The cells are allowed to grow for a defined period of time. For example, depending on the starting concentration of the cell culture, the temperature at which the cells are grown, and the intrinsic growth rate of the cells, the cells may be grown for 0, 1, 2, 3, 4, 5, 6, 7, 8, 9, 10, 11, 12, 13, 14, 15, 16, 17, 18, 19, 20 or more days. The practitioner of the present invention will be able to choose the duration of growth depending on the polypeptide production requirements and the needs of the cells themselves.

### Monitoring Culture Conditions

In certain embodiments of the present invention, particular conditions of the growing cell culture are monitored. Monitoring cell culture conditions allows for the determination of whether the cell culture is producing recombinant polypeptide or protein at suboptimal levels or whether the culture is about to enter into a suboptimal production stage.

As non-limiting examples, it may be beneficial or necessary to monitor temperature, pH, cell density, cell viability, integrated viable cell density, lactate levels, ammonium levels, osmolarity, or titer of the expressed polypeptide or protein. Numerous techniques are well known in the art that will allow one of ordinary skill in the art to measure these conditions. For example, cell density may be measured using a hemacytometer, a Coulter counter (Vi-Cell), or Cell density examination (CEDEX). Viable cell density may be determined by staining a culture sample with Trypan blue. Since only dead cells take up the Trypan blue, viable cell density can be determined by counting the total number of cells, dividing the number of cells that take up the dye by the total number of cells, and taking the reciprocal. Optionally, cell viability is at least 50%, at least 60%, at least 70%, at least 80% or at least 90% over the whole production culture period. HPLC can be used to determine the levels of lactate, ammonium or the expressed polypeptide or protein. Alternatively, the level of the expressed polypeptide or protein can be determined by standard molecular biology techniques such as coomassie staining of SDS-PAGE gels, Western blotting, Bradford assays, Lowry assays, Biuret assays, and UV absorbance. It may also be beneficial or necessary to monitor the post-translational modifications of the expressed polypeptide or protein, including phosphorylation and glycosylation.

### Isolation of Expressed Polypeptide

In general, it will typically be desirable to isolate and/or purify proteins or polypeptides expressed according to the present invention. In one embodiment, the expressed polypeptide or protein is secreted into the medium and thus cells and other solids may be removed, as by centrifugation or filtering for example, as a first step in the purification process. This embodiment is particularly useful when used in accordance with the present invention, since the methods and compositions described herein result in increased cell viability. As a result, fewer cells die during the culture process, and fewer proteolytic enzymes are released into the medium which can potentially decrease the yield of the expressed polypeptide or protein.

### Recombinant Polypeptides

The methods of the present invention can be used for large-scale production of any recombinant polypeptides of interest, including therapeutic antibodies. Non-limiting examples of recombinant polypeptides that can be produced by the methods provided herein include antibodies (including intact immunoglobulins or antibody fragments), enzymes (e.g., a galactosidase), proteins (e.g., human erythropoietin, tumor necrosis factor (TNF), or an interferon alpha or beta), cellular receptors (e.g., EGFR) or immunogenic or antigenic proteins or protein fragments (e.g., proteins for use in a vaccine). Antibodies within the scope of the present invention include, but are not limited to: anti-HER2 antibodies including Trastuzumab (HERCEPTIN®) (Carter et al., Proc. Natl. Acad. Sci. USA, 89:4285-4289 (1992); anti-HER3 antibodies; anti-HER4 antibodies; U.S. Pat. No. 5,725,856); anti-CD20 antibodies such as chimeric anti-CD20 “C2B8” as in U.S. Pat. No. 5,736,137 (RITUXAN®), a chimeric or humanized variant of the 2H7 antibody as in U.S. Pat. No. 5,721,108B1, or Tositumomab (BEXXAR®); anti-IL-8 (St John et al., Chest, 103:932 (1993), and International Publication No. WO 95/23865); anti-VEGF antibodies including humanized and/or affinity matured anti-VEGF antibodies such as the humanized anti-VEGF antibody huA4.6.1 AVASTIN® (Kim et al., Growth Factors, 7:53-64 (1992), International Publication No. WO 96/30046, and WO 98/45331, published Oct. 15, 1998); anti-PSCA antibodies (WO01/40309); anti-CD40 antibodies, including S2C6 and humanized variants thereof (WO00/75348); anti-CD11a (U.S. Pat. No. 5,622,700, WO 98/23761, Steppe et al., Transplant Intl. 4:3-7 (1991), and Hourmant et al., Transplantation 58:377-380 (1994)); anti-IgE (Presta et al., J. Immunol. 151:2623-2632 (1993), and International Publication No. WO 95/19181); anti-CD18 (U.S. Pat. No. 5,622,700, issued Apr. 22, 1997, or as in WO 97/26912, published Jul. 31, 1997); anti-IgE (including E25, E26 and E27; U.S. Pat. No. 5,714,338, issued Feb. 3, 1998 or U.S. Pat. No. 5,091,313, issued Feb. 25, 1992, WO 93/04173 published Mar. 4, 1993, or International Application No. PCT/US98/13410 filed Jun. 30, 1998, U.S. Pat. No. 5,714,338); anti-Apo-2 receptor antibody (WO 98/51793 published Nov. 19, 1998); anti-TNF-α antibodies including cA2 (REMICADE®), CDP571 and MAK-195 (See, U.S. Pat. No. 5,672,347 issued Sep. 30, 1997, Lorenz et al., J. Immunol. 156(4):1646-1653 (1996), and Dhainaut et al., Crit. Care Med. 23(9):1461-1469 (1995)); anti-Tissue Factor (TF) (European Patent No. 0 420 937 B1 granted Nov. 9, 1994); anti-human α₄β₇ integrin (WO 98/06248 published Feb. 19, 1998); anti-EGFR (chimerized or humanized 225 antibody as in WO 96/40210 published Dec. 19, 1996); anti-CD3 antibodies such as OKT3 (U.S. Pat. No. 4,515,893 issued May 7, 1985); anti-CD25 or anti-tac antibodies such as CHI-621 (SIMULECT®) and (ZENAPAX®) (See U.S. Pat. No. 5,693,762 issued Dec. 2, 1997); anti-CD4 antibodies such as the cM-7412 antibody (Choy et al., Arthritis Rheum 39(1):52-56 (1996)); anti-CD52 antibodies such as CAMPATH-1H (Riechmann et al., Nature 332:323-337 (1988)); anti-Fc receptor antibodies such as the M22 antibody directed against FcγRI as in Graziano et al., J. Immunol. 155(10):4996-5002 (1995); anti-carcinoembryonic antigen (CEA) antibodies such as hMN-14 (Sharkey et al., Cancer Res. 55(23 Suppl): 5935s-5945s (1995); antibodies directed against breast epithelial cells including huBrE-3, hu-Mc 3 and CHL6 (Ceriani et al., Cancer Res. 55(23): 5852s-5856s (1995); and Richman et al., Cancer Res. 55(23 Supp): 5916s-5920s (1995)); antibodies that bind to colon carcinoma cells such as C242 (Litton et al., Eur J. Immunol. 26(1):1-9 (1996)); anti-CD38 antibodies, e.g. AT 13/5 (Ellis et al., J. Immunol. 155(2):925-937 (1995)); anti-CD33 antibodies such as Hu M195 (Jurcic et al., Cancer Res 55(23 Suppl): 5908s-5910s (1995) and CMA-676 or CDP771; anti-CD22 antibodies such as LL2 or LymphoCide (Juweid et al., Cancer Res 55(23 Suppl): 5899s-5907s (1995)); anti-EpCAM antibodies such as 17-1A (PANOREX®); anti-GpIIb/IIIa antibodies such as abciximab or c7E3 Fab (REOPRO®); anti-RSV antibodies such as MEDI-493 (SYNAGIS®); anti-CMV antibodies such as PROTOVIR®; anti-HIV antibodies such as PRO542; anti-hepatitis antibodies such as the anti-Hep B antibody OSTAVIR®; anti-CA 125 antibody OvaRex; anti-idiotypic GD3 epitope antibody BEC2; anti-αvβ3 antibody VITAXIN®; anti-human renal cell carcinoma antibody such as ch-G250; ING-1; anti-human 17-1A antibody (3622W94); anti-human colorectal tumor antibody (A33); anti-human melanoma antibody R24 directed against GD3 ganglioside; anti-human squamous-cell carcinoma (SF-25); anti-human leukocyte antigen (HLA) antibodies such as Smart ID10; anti-PD-1 antibodies; anti-PD-L1 antibodies; anti-LAG-3 antibodies; anti-GITR antibodies; anti-TIGIT antibodies; anti-CXCR4 antibodies; anti-CD73 antibodies; and the anti-HLA DR antibody Oncolym (Lym-1).

The foregoing description is to be understood as being representative only and is not intended to be limiting. Alternative methods and materials for implementing the invention and also additional applications will be apparent to one of skill in the art, and are intended to be included within the accompanying claims.

### EXAMPLES

### Example 1

### Cell Lines and Media

CHOK1 GS cell lines (i.e., cell line A, cell line B and cell line C) producing 3 mAbs (i.e., mAb1, mAb2, and mAb3) were used in these experiments. The basal and feed media used were chemically-defined as shown in Table 1. The seed media were equal to the basal medium plus different amounts of L-methionine sulfoximine (MSX), of which 25 μM MSX was added into the seed media for cell line A and C, while 6.25 μM MSX was added into the seed media for cell line B.

TABLE 1
Medium nutrient concentrations for the example 1

| | Total Amino | Glucose | Osmolality |
|---|---|---|---|
| Medium | Acids (mM) | (g/L) | (mOsm/kg) |
| Basal medium | 72 | 6 | 305 |
| 8% enriched basal medium | 98 | 8.5 | 360 |
| 16% enriched basal medium | 126 | 11 | 415 |
| Feed medium | 400 | 78 | 1450 |

### Analyses

For production cultures, the mAb titer was measured using Protein A UPLC. The titer was then normalized relative to the maximum final titer reached by all the 3 CHO cell lines in this study. The normalized titer is expressed as normalized weight/L.

### N−1 Seed Cultures

N−1 cultures were grown in batch mode and centrifuged to simulate the high density N−1 seed culture needed for increased production inoculation cell density. The mAb1 and mAb3 producing seed cultures were grown in BMS proprietary media with 25 μM MSX as selection agent. The mAb2 producing seed cultures were grown in BMS proprietary media with 6.25 μM MSX as selection agent. Batch mode N−1 seed cultures were centrifuged and resuspended in spent media to produce a cell density of 24×10⁶ cells/mL. A portion of the 24×10⁶ cells/mL cell cultures were diluted two-fold with spent media to produce seed cultures at 12×10⁶ cells/mL. Separate portions of the 24×10⁶ cells/mL cell culture were diluted twelve-fold with spent media to produce seed cultures at 2×10⁶ cells/mL. The 2, 12, and 24×10⁶ cells/mL were used to inoculate 0.5, 3, and 6×10⁶ cells/mL production inoculation cell density conditions respectively. There was a four-fold dilution of cells from the N−1 cultures to the production cultures for each production cell density tested.

### Production Cultures

High-throughput screening in 50-mL TubeSpin® bioreactors was used to research the effect of intensified inoculation density on fed-batch product titer for three mAb producing CHO cell lines. High-throughput screening with TubeSpin® bioreactors was applied as an effective scale-down model to simultaneously test many different conditions. The experimental design utilized was a 3×3×3×3 I-Optimal custom Design of Experiments (DOE) generated in SAS JMP Version 13 with 32 conditions per mAb. Main effects, second-order polynomials, and all interactions were included as model terms for the design generation. Factors screened were inoculation density (0.5, 3 or 6×10⁶ cells/mL), level of basal medium enrichment with the feed nutrients (adding 0%, 8% or 16% of the feed nutrients to the basal medium), day of first feed addition (day 1, 2 or 3), and amount of feed per bolus (3.1%, 3.6% or 4.1% of current culture volume).

All TubeSpin® bioreactors used an 18 mL initial working volume on a shaker incubator at 300 rpm and 36.5° C. with 5% CO₂. Temperature was shifted to 33° C. on day 6 of culture. Daily sampling and feeding was carried out automatically using a Tecan liquid handler. Samples were collected on day 14 of culture and analyzed for titer. Stepwise regression was applied to the final titer data to screen for the most significant factors and interactions. A standard least squares fit model was generated for each mAb.

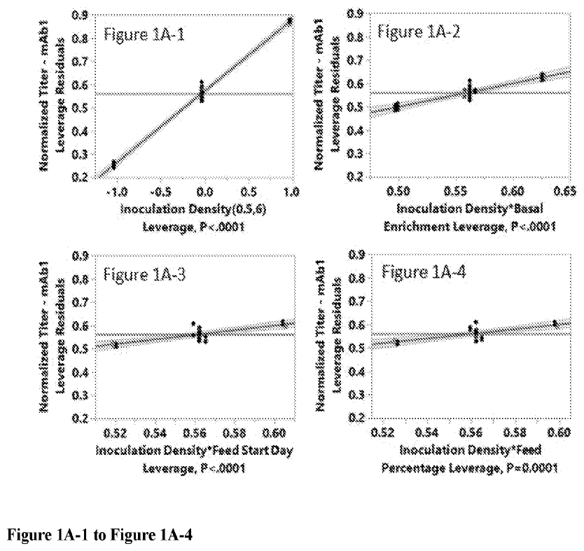

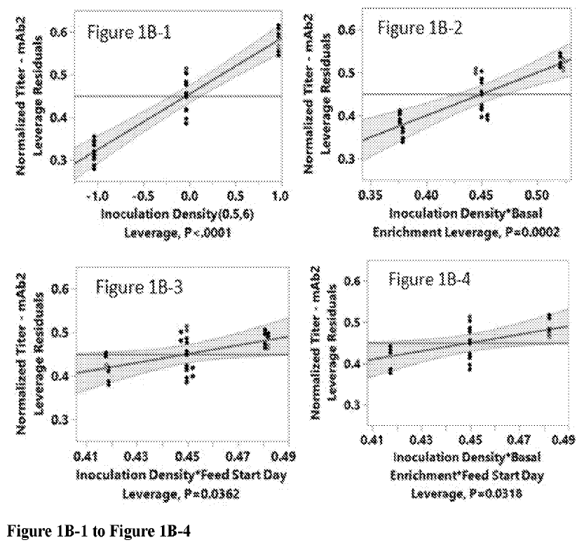

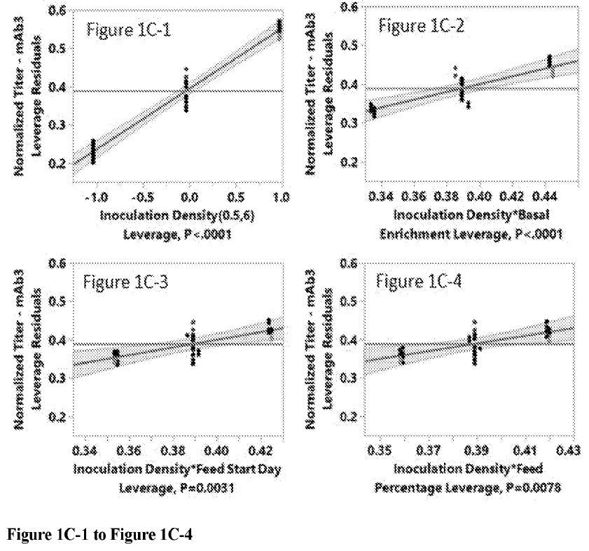

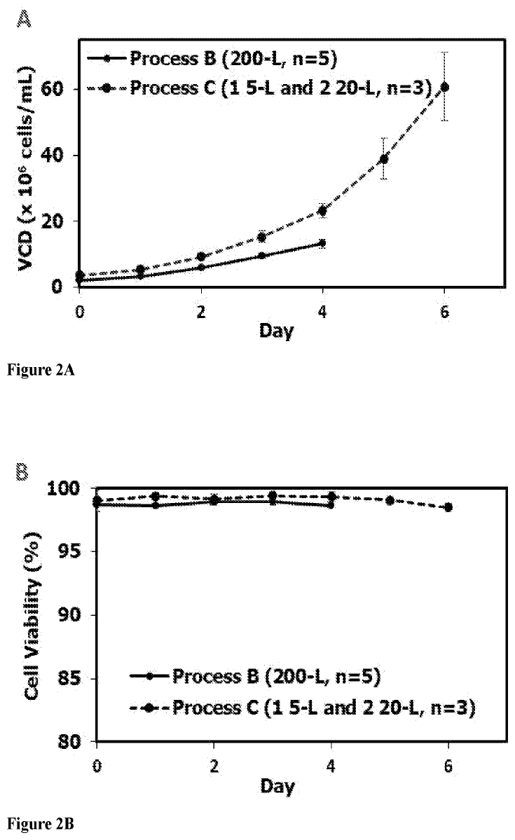

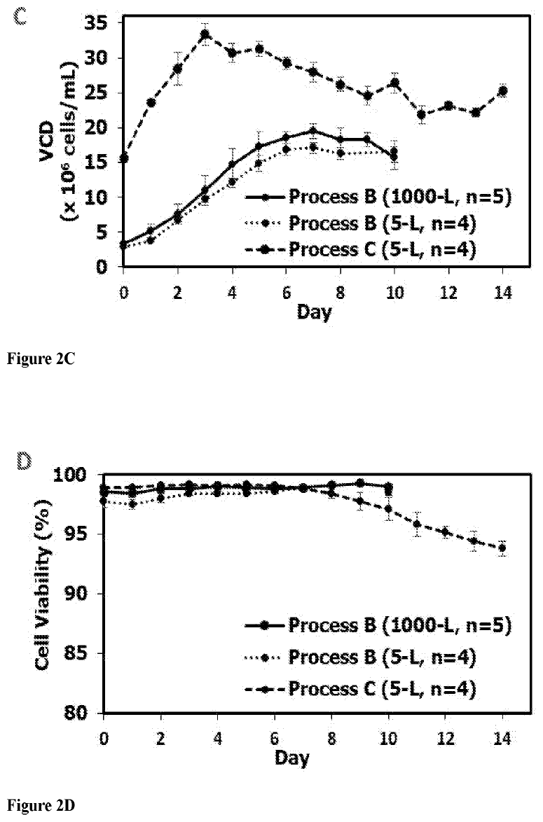

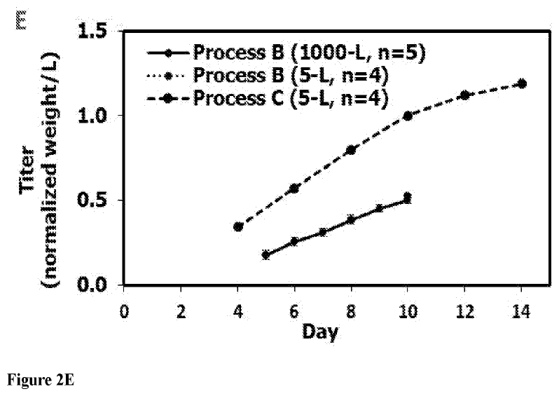

FIG. 1 describes the effects of inoculation density, basal enrichment, feed start day and feed percentage on product titer by SAS JMP data analysis in high-throughput screening cell cultures run using 96 50-mL TubeSpin bioreactors with 32 conditions for each cell line (n=1). FIG. 1A shows cell line A data for mAb1 production. FIG. 1A-1 shows that mAb1 titer was significantly increased by increasing inoculation cell densities from 0.5, 3 and 6×10⁶ cells/mL (P<0.0001). FIG. 1A-2 shows that there was a significant interaction between inoculation cell density and basal medium enrichment on mAb1 production titer (P<0.0001). The more enriched media resulted in higher titer for higher inoculation cell density. FIG. 1 A-3 shows that there was a significant interaction between inoculation cell density and feed start day on mAb1 production titer (P<0.0001). The earlier feeding day resulted in higher titer for higher inoculation cell density. FIG. 1A-4 shows that there was a significant interaction between inoculation cell density and feed percentage on mAb1 production titer (P=0.0001). The higher feed percentage resulted in higher titer for higher inoculation cell density. FIG. 1 B shows cell line B data for mAb2 production. FIG. 1 B-1 shows that mAb2 titer was significantly increased by increasing inoculation cell densities from 0.5, 3 and 6×10⁶ cells/mL (P <0.0001). FIG. 1 B-2 shows that there was a significant interaction between inoculation cell density and basal medium enrichment on mAb2 production titer (P=0.0002). The more enriched media resulted in higher titer for higher inoculation cell density. FIG. 1 B-3 shows that there was a significant interaction between inoculation cell density and feed start day on mAb2 production titer (P=0.0362). The earlier feeding day resulted in higher titer for higher inoculation cell density. FIG. 1 B-4 shows that there was a significant interaction among inoculation cell density, basal enrichment, and feed start day on mAb2 production titer (P=0.0318). The earlier feed start day and higher feed percentage resulted in higher titer for higher inoculation cell density. FIG. 1 C shows cell line C data for mAb3 production. FIG. 1 C-1 shows that mAb3 titer was significantly increased by increasing inoculation cell densities from 0.5, 3 and 6×10⁶ cells/mL (P<0.0001). FIG. 1 C-2 shows that there was a significant interaction between inoculation cell density and basal medium enrichment on mAb3 production titer (P<0.0001). The more enriched media resulted in higher titer for higher inoculation cell density. FIG. 1 C-3 shows that there was a significant interaction between inoculation cell density and feed start day on mAb3 production titer (P=0.0031). The earlier feeding day resulted in higher titer for higher inoculation cell density. FIG. 1 C-4 shows that there was a significant interaction between inoculation cell density and feed percentage on mAb3 production titer (P=0.0078). The higher feed percentage resulted in higher titer for higher inoculation cell density.

### Example 2

### Cell Lines and Media

CHOK1 GS cell line producing mAb1 was used in the experiment. The basal and feed media used were chemically-defined as shown in Table 2. For mAb1 Process B, the seed medium from vial thaw to N−2 stage was equal to the basal medium in Table 1 supplemented with 25 μM of MSX, and N−1 stage seed medium was enriched basal medium supplemented with 25 μM MSX. For mAb1 Process C, the seed medium was equal to the basal medium in Table 1 supplemented with 25 μM of MSX and additional 2 g/L of glucose.

TABLE 2
Medium nutrient concentrations for the example 2

| | | Total Amino | Glucose | Osmolality |
|---|---|---|---|---|
| Medium | Process | Acids (mM) | (g/L) | (mOsm/kg) |
| Basal medium | Process B | 118 | 11 | 410 |
| | Process C | 137 | 10 | 425 |
| Feed medium | Process B | 446 | 78 | 1575 |
| | Process C | 650 | 156 | 2100 |

### Analyses

Samples were taken daily for offline measurement of viable cell density (VCD), cell viability, pH, pCO₂, pO₂, and key metabolites including glucose, glutamine, glutamate, lactate, and ammonium. VCD and viability were measured using a Vi-Cell automated cell counter (Beckman Coulter). pH, pCO₂, pO₂ were measured using a BioProfile pHOX (Nova Biomedical). Metabolites were analyzed using a Cedex Bio HT (Roche). For perfusion N−1, VCD was also measured online using an Incyte biomass capacitance probe (Hamilton). For production culture, a Protein A UPLC method was used to measure protein titer every two days, starting from Day 6 until the end of run. The titer was then normalized relative to mAb1 Process C Day 10 titer. The normalized titer is expressed as normalized weight/L.

### N−1 Seed Cultures

N−1 seed cultures were operated in the batch mode and the perfusion mode. Batch N−1 cultures involved growing the cells in 200-L bioreactors. Perfusion N−1 cultures involved growing the cells in 5-L or 20-L bioreactors. An auxiliary ATF-2 (Repligen) was connected to the bioreactor to perfuse the culture. Fresh culture medium (1× concentrated) is continuously added while old culture medium is continuously removed at the same rate. The perfusion rate is a function of VCD as measured by an online capacitance probe (Hamilton).

The N−1 culture was initiated at a seed density of 3.5×10⁶ cells per mL for a duration of 6 days. Perfusion was started on day 1 at a rate of 0.04 nL/cell/day. Dissolved oxygen (DO) was maintained at 40% and pH was controlled with between 6.6 and 7.6. Temperature was maintained at 36.5° C. The peak VCD reached 60×10⁶ cells per mL on Day 6, and cell viability maintained above 95% over the entire culture period.

### Production Cultures

For both Process B and Process C, the production cultures were grown in the fed-batch mode. Process B production bioreactors were at 5-L scale with 3 L initial working volume and 1000-L scale with 700 L initial working volume, while process C was at 5-L scale with 3-L initial working volume. During the entire course of the run, dissolved oxygen (DO) was maintained at 40% and pH was controlled between 6.6 and 7.6. The process parameters are summarized in Table 3.

TABLE 3
Process Parameter Changes in Process C for mAb1 production

| Process | B | C |
|---|---|---|
| N-1 seed | Batch in 200-L | Perfusion in 5-L |
| | | and 20-L |
| Fed-batch | 5-L and 1000-L | 5-L |
| production | | |
| SD | 3.0 | 15 |
| (×10⁶ cells/mL) | | |
| Feed rate | Fixed at 3.5% of current | Fixed at 4% of current |
| | volume from D 3 on | volume from D 1 on |
| Temperature shift to | 34° C. on D 6 | 34° C. on D 4 |

FIG. 2 shows the comparison of Process B in 5-L and 1000-L bioreactors and Process C in 5-L bioreactors for mAb1 production. The media and process changes are shown in Table 2 and Table 3, respectively. FIG. 2A shows viable cell density (VCD) profiles and FIG. 2B shows cell viability profiles for the enriched N−1 seed at 200-L scale for Process B and the perfusion N−1 seed at lab scales for Process C. The N−1 seed culture for Process C reached much higher final VCD because of perfusion, while the cell viability profiles were similar. FIG. 2C shows VCD profiles, FIG. 2D shows viability profiles and FIG. 2E shows titer profiles at 5-L and 1000-L scales using the same 200-L N−1 batch seed for Process B, and at 5-L scale using the perfusion seed for Process C. The VCD in fed-batch production for Process C was much higher than the VCD in the fed-batch production for Process B. Although the viability for Process C was slightly lower than Process B, the viability for both processes remained high (above 95%) until day 10. Process C achieved approximately doubled titer of Process B for the entire culture duration. The increased titer and volumetric productivity in Process C were mainly attributed to significantly higher VCD in the production stage, since the cell specific productivities were comparable between Process B and Process C (FIG. 2E and Table 4).

TABLE 4
Cell culture performance summary for Process B and
Process C for mAb1 production. The Process C titer
at 5-L scale on day 10 was normalized as 1 for mAb1.

| | | | Cell specific | |
|---|---|---|---|---|
| | | | productivity | Volumetric |
| | Normalized | Peak VCD | (normalized | productivity |
| | Titer | (×10⁶ | weight/ | (normalized |
| | (weight/L) | cells/mL) | cell/day) | weight/L/day) |
| Process B | 0.50 ± 0.02 | 19.5 ± 1.1 | 3.03 ± 0.21 | 0.042 ± 0.002 |
| at 1000-L | | | | |
| (n = 5) | | | | |
| Process C | 1.00 ± 0.01 | 33.4 ± 1.6 | 3.04 ± 0.07 | 0.084 ± 0.001 |
| at 5-L | | | | |
| (n = 4) | | | | |

### Example 3

### Cell Lines and Media

CHOK1 GS cell line producing mAb2 was used in the experiment. The basal and feed media used were chemically-defined as shown in Table 5. The seed medium used during vial thaw and seed culture steps for all the processes was the same formulation, and was equal to the basal medium in Example 1 plus 6.25 μM L-methionine sulfoximine (MSX).

TABLE 5
Medium nutrient concentrations for the example 3

| | | Total Amino | Glucose | Osmolality |
|---|---|---|---|---|
| Medium | Process | Acids (mM) | (g/L) | (mOsm/kg) |
| Basal medium | Process A | 69 | 6 | 300 |
| | Process B | 99 | 10 | 380 |
| | Process C | 137 | 10 | 425 |
| Feed medium | Process A | 344 | 60 | 1310 |
| | Process B | 404 | 78 | 1450 |
| | Process C | 404 | 110 | 1570 |

### Analyses

Samples were taken daily for offline measurement of viable cell density (VCD), cell viability, pH, pCO₂, pO₂, and key metabolites including glucose, glutamine, glutamate, lactate, and ammonium. VCD and viability were measured using a Vi-Cell automated cell counter (Beckman Coulter). pH, pCO₂, pO₂ were measured using a BioProfile pHOX (Nova Biomedical). Metabolites were analyzed using a Cedex Bio HT (Roche). For perfusion N−1, VCD was also measured online using an Incyte biomass capacitance probe (Hamilton). For production culture, a Protein A UPLC method was used to measure protein titer every two days, starting from Day 6 until the end of run. The titer was then normalized relative to the day 14 titer for Process C at 500-L scale in Example 4. The normalized titer is expressed as normalized weight/L.

### N−1 Seed Cultures

For Process A, N−1 was operated in the batch mode in 2-L shake flask with a working volume of 1 L. The shake flask seed was cultivated in a humidified incubator (Climo-Shaker, Kuhner) using standard conditions of 36.5° C., 5% CO₂ and proper agitation. The N−1 was initiated at a seed density of 0.5×10⁶ cells per mL for a duration of 4 days. The final VCD reached above 6×10⁶ cells/mL with over 99% cell viability on day 4.

For Process B and C, N−1 was operated in the perfusion mode. Perfusion N−1 cultures involved growing the cells in 5-L bioreactors with a working volume of 3.5 L. An auxiliary ATF-2 (Repligen) was connected to the bioreactor to perfuse the culture. Fresh culture medium (lx concentrated) is continuously added while old culture medium is continuously removed at the same rate. The perfusion rate is a function of VCD as measured by an online capacitance probe (Hamilton).

The perfusion N−1 culture was initiated at a seed density of 3.2×10⁶ cells per mL for a duration of 6 days. Perfusion was started on day 1 at a rate of 0.04 nL/cell/day. Dissolved oxygen (DO) was maintained at 40% and pH was controlled between 6.8 and 7.6. Temperature was maintained at 36.5° C. The peak VCD reached above 90×10⁶ cells per mL on Day 6, and cell viability maintained above 90% over the entire culture period.

### Production Cultures

The production cultures were grown in the fed-batch mode in 5-L bioreactor with an initial working volume of 3 L. During the entire course of the run, dissolved oxygen (DO) was maintained at 40% and pH was controlled between 6.8 and 7.6. Three sets of basal and feed media were evaluated at four different seeding densities of 1.5×10⁶, 3×10⁶, 11×10⁶, and 19×10⁶ cells/mL, respectively. For basal and feed, Process B basal and feed are composed of the same base formulation as Process A basal and feed, respectively, but are further enriched with higher concentrations of nutrients, while the base media formulations used in Process C were rebalanced from Process B media, including addition or removal of some components and increasing or decreasing concentrations for others.

For all the conditions, the production culture has a duration of 14 days, with dissolved oxygen (DO) maintained at 40%, pH controlled between 6.8 and 7.6, and a daily feed at a feeding weight of 3.6% of initial culture weight. For the production culture that was seeded at 1.5×10⁶ cells/mL (Condition 1), daily feed was started on day 3, and temperature was maintained at 36.5° C.; for the production cultures that were seeded at 3.0×10⁶ cells/mL (Condition 2 and 5), daily feed was started on day 4, and temperature was maintained at 36.5° C. and shifted to 32° C. on day 5; for the production cultures that were seeded at 11.0×10⁶ cells/mL (Condition 3 and 6), daily feed was started on day 2, and temperature was maintained at 36.5° C. and shifted to 32° C. on day 4; for the production cultures that were seeded at 19.0×10⁶ cells/mL (Condition 4 and 7), daily feed was started on day 1, and temperature was maintained at 36.5° C. and shifted to 32° C. on day 3. Details on the process parameters of different conditions are listed in Table 6.

TABLE 6
Experimental design for the study of effects of inoculation cell densities
and media on mAb2 production in fed-batch 5-L bioreactors (n = 2)

| | | Conditions | | | | | | |
|---|---|---|---|---|---|---|---|---|
| Parameters | | 1 | 2 | 3 | 4 | 5 | 6 | 7 |
| Inoculum | Inoculation | 1.5 | 3 | 11 | 19 | 3 | 11 | 19 |
| | seeding density | | | | | | | |
| | (SD) | | | | | | | |
| | (×10⁶ cells/mL) | | | | | | | |
| Media | Basal | Process A | Process B | | | Process C | | |
| | Feed | Process A | Process B | | | Process C | | |
| Feeding | Daily feed rate | 3.6% of initial volume | | | | | | |
| strategy | Feed start day | 3 | 4 | 2 | 1 | 4 | 2 | 1 |
| Bioreactor | Temperature | NA | Day 5 | Day 4 | Day 3 | Day 5 | Day 4 | Day 3 |
| condition | shift to 32° C. | | | | | | | |

FIG. 3 shows the effects of inoculation cell density or seeding density (SD) and media change on cell culture performance for mAb2 production in 5-L fed-batch bioreactors. The experimental design is shown in Table 6. FIG. 3A shows VCD profiles for different conditions (Table 6). The higher inoculation density gave a higher peak VCD with the same basal and feed media. For the same inoculation density, Process B media condition had higher peak VCD than Process C media condition. FIG. 3B shows titer profiles for different conditions (Table 6). Process B and C gave much higher titer than Process A at all the inoculation densities tested in the study. For Process B and C comparison, increasing inoculation density did not improve final titer significantly with Process B media (Solid lines), while increasing inoculation density improved final titer significantly with Process C media (dashed lines). The change of media from Process B (solid triangle line) to Process C only (dashed triangle line) slightly improved titer for the control inoculation density at 1.5×10⁶ cells/mL, while the conditions (dashed square line and dashed circle line) with both increasing inoculation cell density and change from Process B to Process C media resulted in significant titer improvement.

### Example 4

### Cell Lines and Media

CHOK1 GS cell line producing mAb2 was used in the experiment. The basal and feed media used for Process B and C in this example were the same formulations as used for Process B and C, respectively, in Example 3 as shown in Table 5. For Process B, the seed medium used at N−1 stage was equal to its basal medium, while the seed medium used during vial thaw and seed passages prior to N−1 was equal to the basal medium in Example 1 plus 6.25 μM L-methionine sulfoximine (MSX). For Process C, the seed medium used during vial thaw and all seed culture steps was the same formulation, and was equal to the basal medium in Example 1 plus 6.25 μM L-MSX. It should be noted that although Example 3 and 4 use the same mAb2, the detailed process parameters for the process with the same name could be different (mainly for Process B N−1 step) due to the different study purposes in the two examples.

### Analyses

Samples were taken daily for offline measurement of VCD, cell viability, pH, pCO₂, pO₂, and key metabolites including glucose, glutamine, glutamate, lactate, and ammonium. VCD and viability were measured using a Vi-Cell automated cell counter (Beckman Coulter). pH, pCO₂, pO₂ were measured using a BioProfile pHOX (Nova Biomedical). Metabolites were analyzed using a Nova Flex 2 (Nova Biomedical) for Process A samples, or a Cedex Bio HT (Roche) for Process B samples, for facility fit. For perfusion N−1, VCD was also measured online using an Incyte biomass capacitance probe (Hamilton).

For production culture, a Protein A UPLC method was used to measure protein titer every two days, starting from Day 4 or Day 6 until the end of run. The titer was then normalized relative to the day 14 titer for Process C at 500-L scale in this example. The normalized titer is expressed as normalized weight/L. Normalized product production rate is calculated as the final normalized titer divided by total duration, expressed as normalized weight/L/day. Overall normalized cell specific productivity (Qp), expressed as normalized weight/cell/day, is calculated as normalized titer divided by the integral of VCD for entire duration.

Size exclusion chromatography (SEC) for high molecular weight (HMW) was performed using a Waters Acquity BEH200 SEC, 4.6 mm ID ×150 mm, 1.7 um, with an isocratic gradient monitored at 280 nm on a Waters Acquity UPLC System with UV Detector (Milford, Mass.) equipped with a temperature controlled autosampler.

Charge Variants were assayed by Imaged Capillary Isoelectric Focusing (iCIEF), which was performed on a Protein Simple iCE3 instrument with an Alcott 720NV autosampler (San Jose, Calif.). Samples were mixed with appropriate pI markers, ampholytes, and urea and injected into a fluorocarbon coated capillary cartridge. A high voltage was applied and the charged variants migrated to their respective pI. A UV camera captured the image at 280 nM. The main peak was identified and the peaks that migrated into the acidic range and basic range were summed, quantitated, and reported as relative percent area.

N-Glycans analysis was performed using a commercially available kit from Waters, GlycoWorks RapiFluor-MS N-Glycan Kit (Milford, Mass.). The free oligosaccharides were profiled using an Acquity UPLC Glycan BEH Amide, 130 Å, 1.7 μm, 2.1×10 mm column (Milford, Mass.) on a Waters Acquity I-Class system (Milford, Mass.) equipped with a temperature controlled autosampler and fluorescence detector.

### N−1 Seed Cultures

For Process B, the N−1 cultures were grown in batch mode. The N−1 bioreactors were at 200-L scale with an initial working volume of 200 L.

For Process C, N−1 was operated in the perfusion mode. Perfusion N−1 cultures involved growing the cells in 200-L disposable bioreactor with an initial volume of 80 L. An auxiliary ATF-6 (Repligen) was connected to the bioreactor to perfuse the culture. Fresh culture medium (lx concentrated) is continuously added while old culture medium is continuously removed at the same rate. The perfusion flow rate is controlled automatically based on the following equation, in which VCD is measure online by an Incyte biomass probe (Hamilton):

Perfusion flow rate (ml/min)=VCD (10⁶ cells/mL)×CSPR (nL/cell/day)×working volume (mL)/1440 (min/day)

The N−1 culture for Process B was initiated at a lower seed density of 1.0×10⁶ cells per mL for 4 days. Dissolved oxygen (DO) was maintained at 40% and pH was controlled between 6.8 and 7.6. Temperature was maintained at 36.5° C. The N−1 culture for Process C was initiated at a higher seed density of 3.2×10⁶ cells per mL for a longer duration of 6 days. Perfusion was started on day 1 at a rate of 0.04 nL/cell/day. Dissolved oxygen (DO) was maintained at 40% and pH was controlled between 6.8 and 7.6. Temperature was maintained at 36.5° C.

Process C had a maximum viable cell density of approximately 91×10⁶ cells per mL on Day 6, while Process B had a maximum viable cell density of approximately 16×10⁶ cells per mL on Day 4. Both N−1 cultures maintained above 90% cell viability over the entire culture period for both Process B and Process C.

### Production Cultures

The production cultures were grown in the fed-batch mode for both Process B and C. The process parameter changes are summarized in Table 7.

TABLE 7
Process parameters for Process B and Process C
for mAb2 production in large scale bioreactors

| Process | B | C |
|---|---|---|
| N-1 seed | Batch in 200-L | Perfusion in 200-L |
| | | and 500-L |
| Fed-batch | 1000-L | 500-L and 2000-L |
| production | | |
| SD | 3.0 | 16 |
| (×10⁶ cells/mL) | | |
| Feed rate | Fixed at 3.1% of initial | Fixed at 4.1% of initial |
| | volume from Day 4 (D 4) | volume from D 2 |
| Temperature shift to | 32° C. on D 5 | 32° C. on D 3 |

Process B production bioreactors were at 1000-L scale with 700 L initial working volume, while Process C was at 500-L scale with 300 L initial working volume and 2000-L scale with 1210-L initial working volume. The basal and feed media used in Process C contain similar nutrient components as those used in Process B, but the concentrations of different nutrients, especially amino acids and salts varied between the two processes.

The production culture for Process B was initiated at a lower seed density of 3.0×10⁶ cells per mL for 14 days. Daily feed was started on day 4 at a feeding weight of 3.1% of initial culture weight per day. Dissolved oxygen (DO) was maintained at 40% and pH was controlled between 6.8 and 7.6. Temperature was maintained at 36.5° C. and shifted to 32° C. on day 5.

The production culture for Process C was initiated at a higher seed density of 16×10⁶ cells per mL for 14 days. Daily feed was started on day 2 at a feeding weight of 4.1% of initial culture weight per day. Dissolved oxygen (DO) was maintained at 40% and pH was controlled between 6.8 and 7.6. Temperature was initially maintained at 36.5° C. and shifted to 32° C. on day 3.

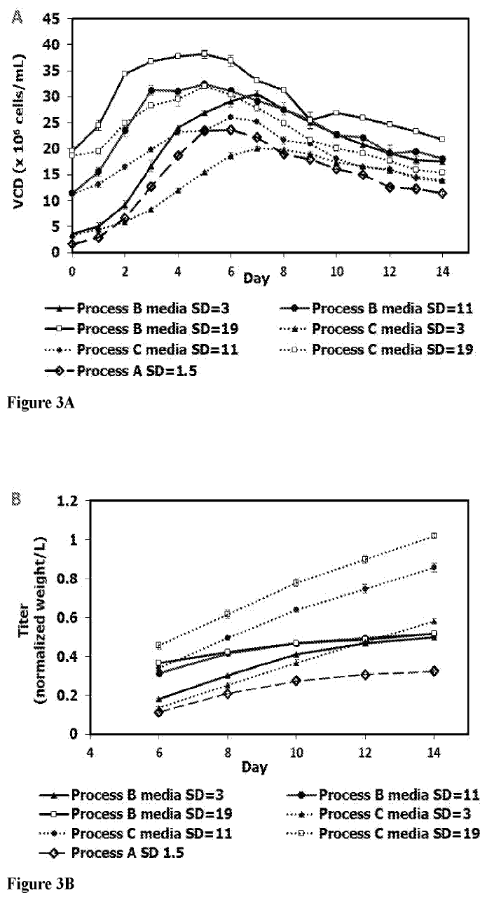

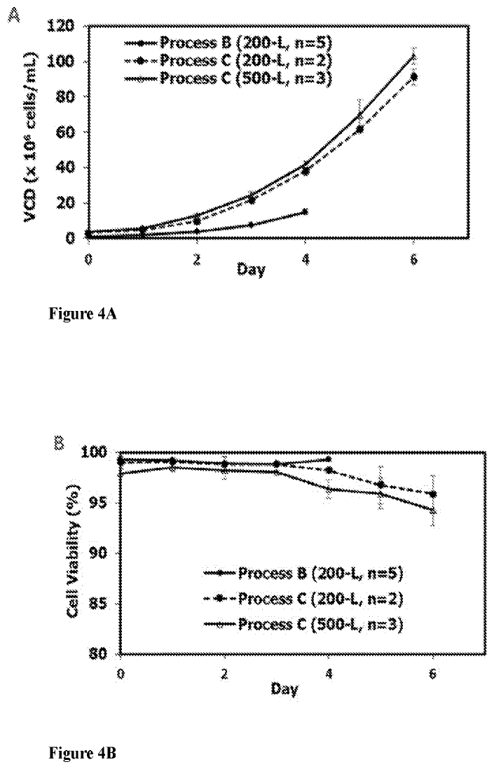

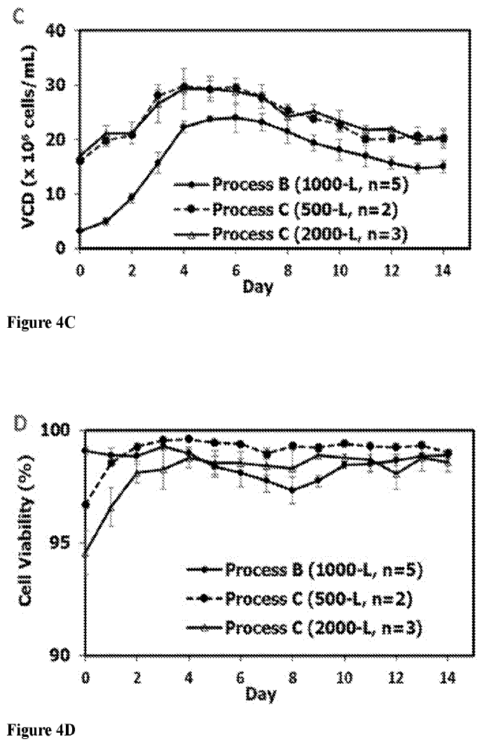

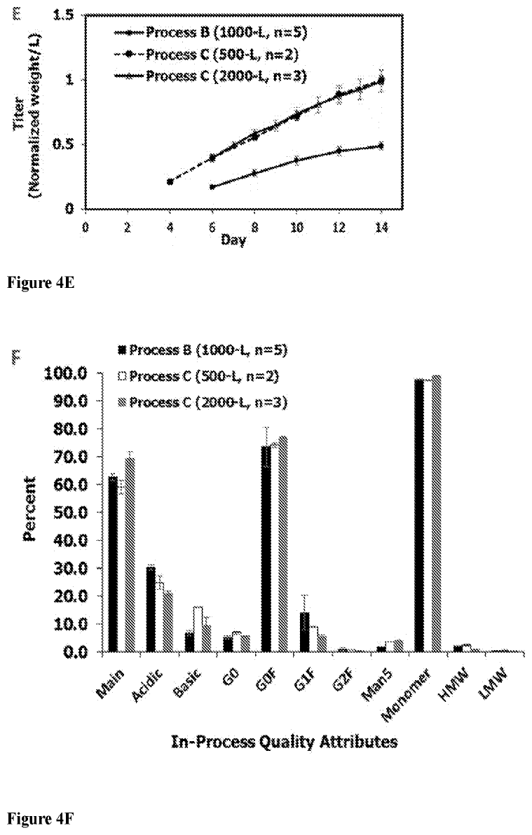

FIG. 4 shows the large scale bioreactor performance of N−1 seed and fed-batch production bioreactors, and the in-process quality attributes for mAb2 Process B and Process C. The media and process parameter changes made from mAb2 Process B to Process C are summarized in Table 5 and Table 7, respectively. For N−1 cultures, the perfusion seed culture achieved a much higher final VCD of about 100×10⁶ cells/mL for Process C when compared with the enriched batch N−1 culture which achieved a final VCD of about 16×10⁶ cells/mL for Process B (FIG. 4A). Due to the high final VCD for the perfusion N−1 seed, the viability dropped to about 95% on day 6, while the enriched batch N−1 seed for Process B maintained cell viability above 99% for the entire 4-day duration (FIG. 4B). In the production bioreactor, the VCD for Process C with 16×10⁶ cells/mL SD was not only much higher in the beginning of culture than the VCD for Process B, but Process C also maintained higher VCD through the entire 14-day duration (FIG. 4C). Due to lower viability at the end of the perfusion N−1 step for Process C (FIG. 4B), the viability for Process C was slightly lower than in Process B at the beginning of the fed-batch production, but the Process C viability increased and trended similarly to Process B from day 2 on (FIG. 4D). There was a viability dip in the middle of the run for Process B, which was not seen for Process C (FIG. 4D). Importantly, the titer for Process C was approximately doubled compared to the titer of Process B throughout the entire 14-day duration (FIG. 4E and Table 8), while in-process quality attributes, e.g. charge variant species, N glycans, and SEC impurities, were similar between Process B and C (FIG. 4F and Table 9). The doubled titer and volumetric productivity for mAb2 Process C compared with Process B can be attributed to both higher VCD and higher cell specific productivity (Table 8).

TABLE 8
Cell culture performance summary for Process
B and Process C for mAb2 production

| | | | Cell specific | |
|---|---|---|---|---|
| | | | productivity | Volumetric |
| | Normalized | Peak VCD | (normalized | productivity |
| | Titer | (×10⁶ | weight/ | (normalized |
| | (weight/L) | cells/mL) | cell/day) | weight/L/day) |
| Process B | 0.49 ± 0.02 | 24.02 ± 2.66 | 2.05 ± 0.19 | 0.04 ± 0.00 |
| at 1000-L | | | | |
| (n = 5) | | | | |
| Process C | 1.00 ± 0.03 | 29.58 ± 0.65 | 2.92 ± 0.06 | 0.07 ± 0.00 |
| at 500-L | | | | |
| (n = 2) | | | | |
| Process C | 0.99 ± 0.09 | 29.33 ± 2.19 | 2.92 ± 0.06 | 0.07 ± 0.00 |
| at 2000-L | | | | |
| (n = 3) | | | | |

TABLE 9
In-process quality attributes for Process B and Process C for mAb2 production

| | | | | | | | | | SEC | SEC | SEC |
|---|---|---|---|---|---|---|---|---|---|---|---|
| | Charge Variants (%) | | | N-linked Glycosylation (%) | | | | | Monomer | HMW | LMW |
| | Main | Acidic | Basic | G0 | G0F | G1F | G2F | Man5 | (%) | (%) | (%) |
| Process | 62.9 ± | 30.4 ± | 6.7 ± | 5.3 ± | 73.6 ± | 14.1 ± | 0.9 ± | 1.9 ± | 97.8 ± | 2.1 ± | 0.1 ± |
| B at | 1.3 | 0.8 | 0.9 | 0.6 | 7.2 | 6.2 | 0.7 | 0.0 | 0.1 | 0.1 | 0.1 |
| 1000-L | | | | | | | | | | | |
| (n = 5) | | | | | | | | | | | |
| Process | 59.1 ± | 24.9 ± | 16.0 ± | 6.9 ± | 74.2 ± | 8.9 ± | 0.5 ± | 3.7 ± | 97.6 ± | 2.5 ± | 0.35 ± |
| C at | 2.4 | 2.3 | 0.2 | 0.5 | 0.7 | 0.1 | 0.0 | 0.0 | 0.1 | 0.3 | 0.4 |
| 500-L | | | | | | | | | | | |
| (n = 2) | | | | | | | | | | | |
| Process | 69.4 ± | 21.1 ± | 9.5 ± | 5.7 ± | 76.9 ± | 5.4 ± | 0.3 ± | 4.0 ± | 99.1 ± | 0.9 ± | 0.1 ± |
| C at | 2.6 | 0.8 | 3.0 | 0.2 | 0.3 | 0.6 | 0.0 | 0.2 | 0.0 | 0.0 | 0.0 |
| 2000-L | | | | | | | | | | | |
| (n = 2) | | | | | | | | | | | |

### Example 5

Cell Lines and Media

CHOK1 GS cell line producing mAb4 was used in these experiments. The basal and feed media used were chemically-defined as shown in Table 10. The seed medium was equal to the basal medium in Example 1 plus 25 μM L-methionine sulfoximine (MSX).

TABLE 10
Medium nutrient concentrations for Example 5

| | | Total Amino | Glucose | Osmolality |
|---|---|---|---|---|
| Medium | Process | Acids (mM) | (g/L) | (mOsm/kg) |
| Basal medium | Process B | 69 | 6 | 300 |
| | Process C | 103 | 6 | 310 |
| Feed medium | Process B | 513 | 80 | 1810 |
| | Process C | 500 | 120 | 2000 |

### Analyses

Samples were taken daily for offline measurement of viable cell density (VCD), cell viability, pH, pCO₂, pO₂, and key metabolites including glucose, glutamine, glutamate, lactate, and ammonium. VCD and viability were measured using a Vi-Cell automated cell counter (Beckman Coulter). pH, pCO₂, pO₂ were measured using a BioProfile pHOX (Nova Biomedical). Metabolites were analyzed using a Nova Flex 2 (Nova Biomedical) for Process B samples, or a Cedex Bio HT (Roche) for Process C samples, for facility fit. For perfusion N−1, VCD was also measured online using an Incyte biomass capacitance probe (Hamilton). For production culture, a Protein A UPLC method was used to measure protein titer every days, starting from Day 4 or Day 5 until the end of run. The titer was then normalized relative to the day 14 titer for Process C at 500-L scale in this example.

Size exclusion chromatography (SEC) for high molecular weight (HMW) was performed using a Waters Acquity BEH200 SEC, 4.6 mm ID×150 mm, 1.7 um, with an isocratic gradient monitored at 280 nm on a Waters Acquity UPLC System with UV Detector (Milford, Mass.) equipped with a temperature controlled autosampler.

Charge Variants were assayed by Imaged Capillary Isoelectric Focusing (iCIEF), which was performed on a Protein Simple iCE3 instrument with an Alcott 720NV autosampler (San Jose, Calif.). Samples were mixed with appropriate pI markers, ampholytes, and urea and injected into a fluorocarbon coated capillary cartridge. A high voltage was applied and the charged variants migrated to their respective pI. A UV camera captured the image at 280 nM. The main peak was identified and the peaks that migrated into the acidic range and basic range were summed, quantitated, and reported as relative percent area.

N-Glycans analysis was performed using a commercially available kit from Waters, GlycoWorks RapiFluor-MS N-Glycan Kit (Milford, Mass.). The free oligosaccharides were profiled using an Acquity UPLC Glycan BEH Amide, 130 Å, 1.7 μm, 2.1×10 mm column (Milford, Mass.) on a Waters Acquity I-Class system (Milford, Mass.) equipped with a temperature controlled autosampler and fluorescence detector.

### N−1 Seed Cultures

For Process B, the N−1 cultures were grown in batch mode. The N−1 bioreactors were at 200-L scale with an initial working volume of 195 L. The seed medium used at N−1 stage contains 25 μM MSX as selection agent, and the same formulation compared to the medium used during vial thaw and seed passages prior to N−1.

For Process C, N−1 was operated in the perfusion mode. The seed medium used at N−1 stage contains 25 μM MSX as selection agent, and has the same formulation as that used during vial thaw and seed passages prior to N−1. Perfusion N−1 cultures involved growing the cells in 200-L disposable bioreactor with an initial volume of 100 L or 200 L. An auxiliary ATF-6 (Repligen) was connected to the bioreactor to perfuse the culture. Fresh culture medium (lx concentrated) is continuously added while old culture medium is continuously removed at the same rate. The perfusion flow rate is controlled automatically based on the following equation, in which VCD is measured online by an Incyte biomass probe (Hamilton):

Perfusion flow rate (ml/min)=VCD (10⁶ cells/mL)×CSPR (nL/cell-day)×working volume (mL)/1440 (min/day)

The N−1 culture for Process B was initiated at a lower seed density of 1.5×10⁶ cells/mL for 4 days. Dissolved oxygen (DO) was maintained at 40% and pH was controlled between 6.7 and 7.5. Temperature was maintained at 36.5° C. The N−1 culture for Process C was initiated at a higher seed density of 4.0×10⁶ cells/mL for a longer duration of 5 days. Perfusion was started on day 0 at a rate of 0.08 nL/cell/day. Dissolved oxygen (DO) was maintained at 40% and pH was controlled between 6.6 and 7.5. Temperature was maintained at 36.5° C.

Process C had a maximum viable cell density of approximately 65×10⁶ cells/mL, while Process B had a maximum viable cell density of approximately 15×10⁶ cells/mL. Both N−1 cultures maintained >95% cell viability over the entire culture period and were similar for Process B and Process C.

### Production Cultures

For both Process B and C as described in Table 11, the production cultures were grown in the fed-batch mode.

TABLE 11
Process parameters for Process B and Process C
for mAb4 production in large scale bioreactors

| Process | B | C |
|---|---|---|
| N-1 seed | Batch in 200-L | Perfusion in 200-L |
| Fed-batch | 1000-L | 500-L |
| production | | |
| SD | 4.5 | 10 |
| (×10⁶ cells/mL) | | |
| Feed rate | 2.6% D 2-3; 3.5% D 4-8; | 2.6% D 1; 3.54% D 2-13; |
| | 2.46% D 9-13 of initial | initial volume |
| | volume | |

Process B production bioreactors were at 1000-L scale with 600 L initial working volume, while Process C at 500-L scale with 300 L initial working volume. The basal and feed media used in Process C contain similar nutrient components as those used in Process B, but the concentrations of different nutrients, especially amino acids, salts and vitamins varied between the two processes.

The production culture for Process B was initiated at a lower seed density of 4.5×10⁶ cells/mL for 14 days. Daily feed was started on day 2 at a feeding weight of 2.6% of initial culture weight, 2.6% on day 3, 3.5% on day 4-8 and 2.46% on day 9-13 based on the initial culture weight. Dissolved oxygen (DO) was maintained at 40% and pH was controlled between 6.7 and 7.5. Temperature was maintained at 36.5° C. during the culture.

The production culture for Process C was initiated at a higher seed density of 10×10⁶ cells/mL for 14 days. Additional phosphate was added on day 0 with a sodium phosphate stock solution. Daily feed was started on day 1 at a feeding weight of 2.6% of initial culture weight on day 1, and 3.54% on the remaining days. Dissolved oxygen (DO) was maintained at 40% and pH was controlled between 6.7 and 7.5. Temperature was maintained at 36.5° C. during the culture.

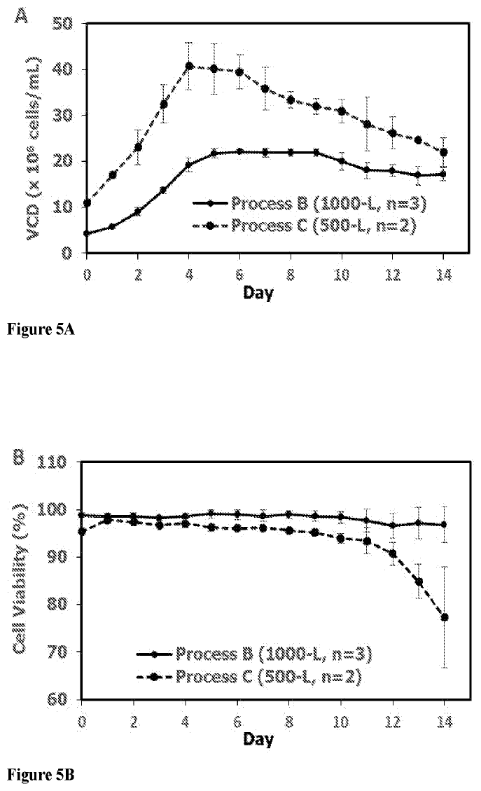

FIG. 5 shows the comparison of mAb4 cell culture performance in large scale bioreactors for Process B and Process C. The media and process parameters for Process B and Process C are shown in Table 10 and Table 11, respectively. FIG. 5A shows VCD profiles for Process B and Process C, described in Table 11. The VCD profile including peak VCD for Process C in 500-L bioreactors were almost doubled over Process B in 1000-L bioreactors (FIG. 5A). The cell viability for Process C was slightly lower than Process B in the end of cell culture (FIG. 5B), titer for Process C was doubled (FIG. 5C), while in-process quality attributes, e.g., charge variant species, N glycans and SEC impurities, were similar between Process B and C (FIG. 5D and Table 13). The two-fold increases in titer and volumetric productivity for Process C were mainly attributed to the much higher VCD compared to Process B, while cell specific productivity was similar between Process B and Process C (FIG. 5D and Table 12).

TABLE 12
Cell culture performance summary for Process
B and Process C for mAb4 production

| | | | Cell specific | |
|---|---|---|---|---|
| | | | productivity | Volumetric |
| | Normalized | Peak VCD | (normalized | productivity |
| | Titer | (×10⁶ | weight/ | (normalized |
| | (weight/L) | cells/ml) | cell/day) | weight/L/day) |
| Process B | 0.53 ± 0.02 | 22.27 ± 0.31 | 2.24 ± 0.05 | 0.034 ± 0.000 |
| (n = 3) | | | | |
| Process C | 1.00 ± 0.05 | 40.89 ± 4.99 | 2.41 ± 0.36 | 0.071 ± 0.001 |
| (n = 2) | | | | |

TABLE 13
In-process quality attributes for Process B and Process C for mAb4 production

| | | | | | | | | | SEC | SEC | SEC |
|---|---|---|---|---|---|---|---|---|---|---|---|
| | Charge Variants (%) | | | N-linked Glycosylation (%) | | | | | Monomer | HMW | LMW |
| | Main | Acidic | Basic | G0 | G0F | G1F | G2F | Man5 | (%) | (%) | (%) |
| Process | 62.3 ± | 35.7 ± | 2.2 ± | 4.6 ± | 63.0 ± | 21.4 ± | 2.8 ± | 2.8 ± | 98.3 ± | 1.6 ± | 0.2 ± |
| B | 0.6 | 0.5 | 0.2 | 0.1 | 1.8 | 1.7 | 0.6 | 0.2 | 0.1 | 0.1 | 0.0 |
| (n = 2) | | | | | | | | | | | |
| Process | 62.1 ± | 36.9 ± | 1.0 ± | 4.2 ± | 70.0 ± | 17.0 ± | 1.9 ± | 2.7 ± | 97.7 ± | 2.1 ± | 0.3 ± |
| C | 2.8 | 2.8 | 0.0 | 0.6 | 1.9 | 1.3 | 0.5 | 0.1 | 0.3 | 0.2 | 0.1 |
| (n = 2) | | | | | | | | | | | |

### Example 6

### Cell Lines and Media

CHOK1 GS cell line producing mAb5 was used in these experiments. The basal and feed media used were chemically-defined as shown in Table 14. The seed medium was equal to the basal medium in Example 1 plus additional glucose and 6.25 μM L-methionine sulfoximine (MSX).

TABLE 14
Medium nutrient concentrations for Example 6

| | | Total Amino | Glucose | Osmolality |
|---|---|---|---|---|
| Medium | Process | Acids (mM) | (g/L) | (mOsm/kg) |
| Basal medium | Process A | 69 | 6 | 300 |
| | Process C | 122 | 10 | 370 |
| Feed medium | Process A | 344 | 60 | 1310 |
| | Process C | 500 | 120 | 2000 |

### Analyses

VCD and cell viability were measured off-line using a Vi-Cell automated cell counter (Beckman Coulter). Culture samples were analyzed off-line using a Nova Flex or Nova Flex 2 for Process A and a Cedex Bio HT (Roche) for Process B to monitor glucose, glutamine, glutamate, lactate, and ammonium. For bioreactor cultures, pH, pCO₂, pO₂ were also measured offline using a BioProfile pHOx (Nova Biomedical). Protein A HPLC or Protein A UPLC methods were used to measure protein titer. The titer was then normalized relative to the day 12 titer for the large-scale (i.e. 500-L) run for Process C.

Size exclusion chromatography (SEC) for high molecular weight (HMW) was performed using a Tosoh Bioscience TSKgel SuperSW3000 column, 4.6 mm×300 mm, 4 μm using an isocratic gradient monitored at 220 nm on a Waters Alliance HPLC system and equipped with temperature controlled autosampler, column heater/cooler, and Waters PDA detector.

Charge Variants were assayed by Cation Exchange High Performance Liquid Chromatography (CEX-HPLC). CEX was performed using a ProPac WCX-10 analytical column, 4 mm×250 mm, 10 μm using a gradient elution (10-100% B) monitored at 280 nm on a Waters Alliance HPLC system equipped with temperature controlled autosampler, column heater/cooler and Waters PDA detector. The main peak was identified and the peaks that migrated into the acidic range and basic range were summed, quantitated, and reported as relative percent area.

Hydrophilic Interaction Liquid Chromatography (HILIC) for N-Glycan analysis was performed using a commercially available kit from Waters, GlycoWorks RapiFluor-MS N-Glycan. This method was performed using a Waters Acquity UPLC Glycan BEH Amide, 130 Å, 2.1 mm×150 mm, 1.7 μm using a gradient elution on a Waters Acquity H-Class UPLC system equipped with temperature controlled autosampler, column heater/cooler and fluorescence detector.

### N−1 Seed Cultures

For Process A, the N−1 cultures were grown in batch mode. Process A N−1 bioreactors were at 200-L scale with 180 L to 200 L initial working volume. For Process C, N−1 was operated in the perfusion mode. Perfusion N−1 cultures involved growing the cells in a 5-L glass bioreactor with an initial volume of 3 L to 4 L or in a 200-L disposable bioreactor with an initial volume of 80 L. An auxiliary ATF-2 (Repligen) or ATF-6 (Repligen) was connected to the bioreactor to perfuse the culture. Fresh culture medium was continuously added while old culture medium was continuously removed. The perfusion flow rate was controlled automatically based on the following equation, in which VCD is measure online by an Incyte biomass probe (Hamilton):

Perfusion flow rate (ml/min)=VCD (10⁶ cells/mL)×CSPR (nL/cell-day)×working volume (mL)/1440 (min/day)

Seed medium for the N−1 stage for Process A and Process C differed only in the concentration of glucose. Process A seed medium had 6 g/L glucose, while Process C seed medium had additional glucose.

The N−1 culture for Process A was initiated at a lower seed density of 1.0×10⁶ cells per mL for 3 days. Dissolved oxygen (DO) was maintained at 40% and pH was controlled between 6.9 and 7.3. Temperature was maintained at 36.5° C.

The N−1 culture for Process C was initiated at a higher seed density of 2.9×10⁶ cells per mL for a longer duration of 6 days. Perfusion was started on day 1 at a rate of 0.04 nL/cell/day. Dissolved oxygen (DO) was maintained at 40% and pH was controlled between 6.6 and 7.4. Temperature was maintained at 36.5° C.

Process C had a maximum viable cell density of approximately 100×10⁶ to 130×10⁶ cells/mL, while Process A had a maximum viable cell density of approximately 7×10⁶ to 9×10⁶ cells/mL. Both N−1 cultures maintained >95% cell viability over the entire culture period and were similar for Process A and Process C.

### Production Cultures

For both Process A and Process C as described in Table 15, the production cultures were grown in fed-batch mode.

TABLE 15
Process parameters for Process A
and Process C for mAb5 production

| Process | A | C |
|---|---|---|
| N-1 seed | Batch | Perfusion |
| Fed-batch | 1000-L | 5-L or 500-L |
| production | | |
| SD | 1.5 | 15 |
| (×10⁶ cells/mL) | | |
| Feed rate | Fixed at 3.6% of initial | Fixed at 4.5% of initial |
| | volume from D 3 | volume from D 1 |
| Temperature shift to | NA | 34° C. on D 4 |

Process A production bioreactors were at 1000-L scale with 630 L initial working volume, while Process C was at 5-L scale with 3 L initial working volume or at 500-L scale with 300 L initial working volume. The basal and feed media used in Process C contain similar nutrient components as those used in Process A, but the concentrations of different nutrients, especially amino acids and salts varied between the two processes. A bolus of sodium phosphate was added on day 0 for Process C.

The production culture for Process A was initiated at a lower seed density of 1.5×10⁶ cells per mL for 14 days. Daily feed was started on day 3 at a feeding volume of 3.6% of initial culture volume per day. Dissolved oxygen (DO) was maintained at 40% and pH was controlled between 6.9 and 7.3. Temperature was maintained at 36.5° C.

The production culture for Process C was initiated at a higher seed density of 15×10⁶ cells per mL for 12 to 14 days. Daily feed was started on day 1 at a feeding volume of 4.5% of initial culture volume per day. Dissolved oxygen (DO) was maintained at 40% and pH was controlled between 6.6 and 7.4. Temperature was initially maintained at 36.5° C. and shifted to 34° C. on day 4.

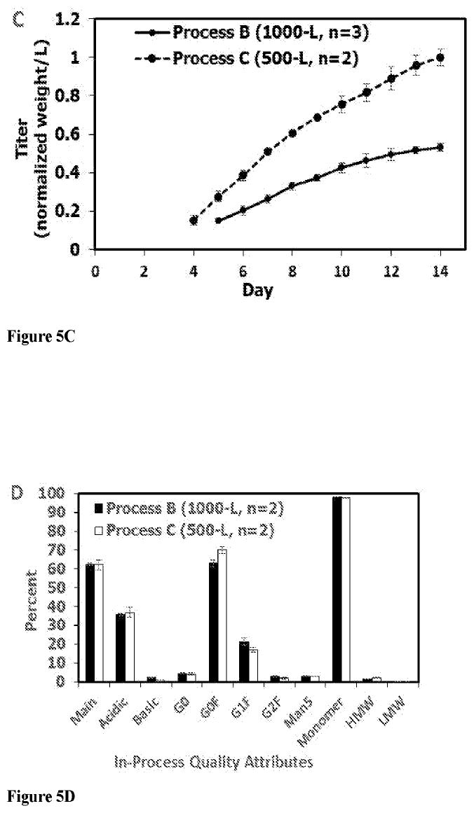

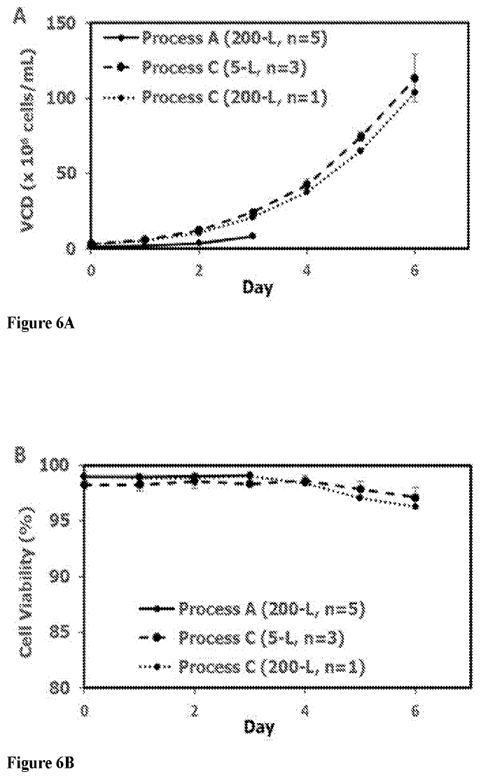

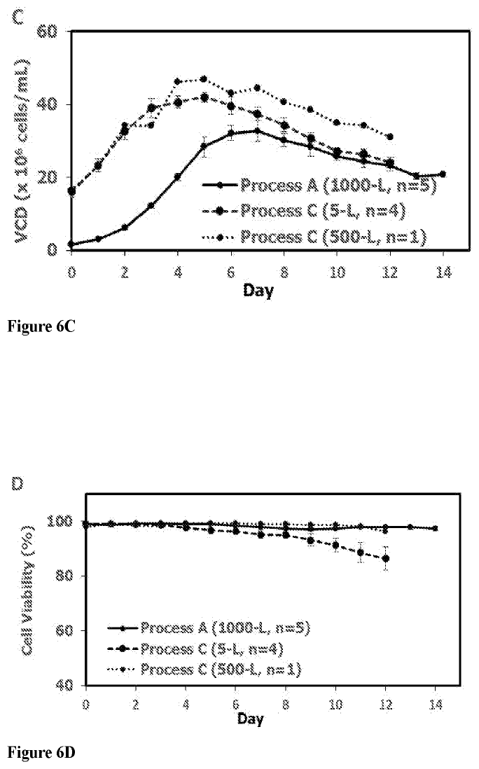

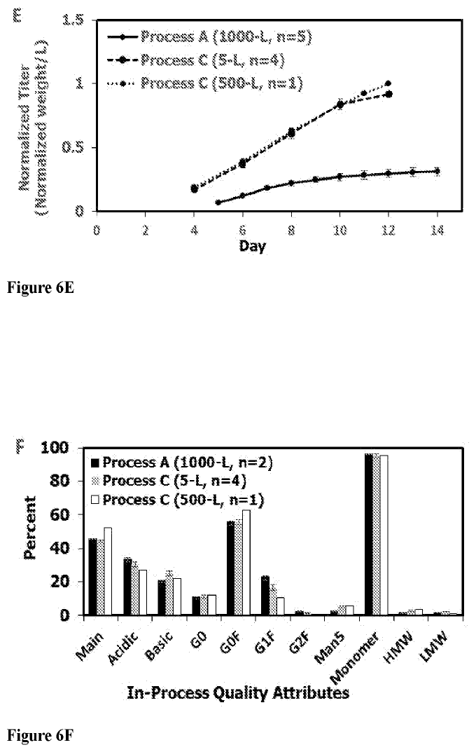

FIG. 6 shows the comparison of mAb5 cell culture performance in 1000-L bioreactors for Process A and in 5-L and 500-L bioreactors for Process C. The media and process parameters for Process A and Process C are shown in Table 14 and table 15, respectively. The N−1 perfusion seed culture reached a final VCD of more than 100×10⁶ cells/mL for Process C, which was more than 10-fold higher than the batch N−1 seed with a final VCD of about 8×10⁶ cells/mL for Process A (FIG. 6A). The final viability Process C was slightly lower than the batch seed (FIG. 6B). Due to the high SD at 15×10⁶ cells/mL, the VCD profile for Process C was significantly higher than Process A with a SD of only 1.5×10⁶ cells/mL, while the 500-L achieved a slightly higher VCD profile than 5-L runs for Process C (FIG. 6C). The cell viability profiles were similar for both Process A at 1000-L and Process C at 500-L for the entire duration, while lower cell viability was observed at 5-L scale (FIG. 6D). Table 16 shows that the higher titer and product production rate for Process C are attributed to higher cell specific productivities and higher peak VCD. The titer was more than tripled from Process A to Process C (FIG. 6E and Table 16), while quality attributes were comparable from Process A to Process C (FIG. 6F).

TABLE 16
Cell culture performance summary for Process
A and Process C for mAb5 production

| | | | Cell specific | |
|---|---|---|---|---|
| | | | productivity | Volumetric |
| | Normalized | Peak VCD | (normalized | productivity |
| | Titer | (×10⁶ | weight/ | (normalized |
| | (weight/L) | cells/mL) | cell/day) | weight/L/day) |
| Process A at | 0.31 ± 0.03 | 32.9 ± 2.7 | 1.05 ± 0.14 | 0.022 ± 0.002 |
| 1000-L on | | | | |
| day 14 | | | | |
| (n = 5) | | | | |
| Process C at | 1.00 ± 0.02 | 42.3 ± 1.1 | 2.28 ± 0.08 | 0.071 ± 0.002 |
| 5-L on | | | | |
| day 14 | | | | |
| (n = 4) | | | | |
| Process C at | 1.00 | 47 | 2.24 | 0.083 |
| 500-L on | | | | |
| day 12 | | | | |
| (n = 1) | | | | |

1. A method of increasing production of a recombinant polypeptide of interest, comprising:
a) seeding mammalian cells in a fed-batch production bioreactor at a viable cell density of at least 5×10⁶ viable cells/ml; and
b) culturing the cells under optimized culture conditions to produce the recombinant polypeptide of interest at high titer.

2. The method of claim 1, wherein the seeding viable cell density is at least 10×10⁶, at least 15×10⁶, at least 20×10⁶, at least 25×10⁶, or at least 30×10⁶ viable cells/ml.

3. The method of claim 1, wherein the cells are cultured in a rebalanced basal medium or an enriched basal medium.

4. The method of claim 1, wherein the cells are fed with a rebalanced feed medium.

5. The method of claim 4, wherein the feed is started at day 1, day 2 or day 3.

6. The method of claim 4, wherein the daily feed percentage is at least 3% of initial culture volume.

7. The method of any one of claims 1-6, wherein the cells are seeded from an N−1 stage perfusion cell culture.

8. The method of any one of claims 1-6, wherein the cells are seeded from an N−1 stage non-perfusion cell culture.

9. The method of any one of claims 1-8, wherein the bioreactor is at least 50 L, at least 500 L, at least 1,000 L, at least 5,000 L, or at least 10,000 L.

10. The method of any one of claims 1-9, wherein the mammalian cells are selected from the group consisting of CHO, VERO, BHK, HEK, HeLa, COS, MDCK and hybridoma cells.

11. The method of claim 10, wherein the mammalian cells are CHO cells.

12. The method of any one of claims 1-11, wherein the recombinant polypeptide of interest is an antibody or antigen-binding fragment.

13. The method of any one of claim 1-12, wherein the antibody or antigen-binding fragment binds an antigen selected from the group consisting of PD-1, PD-L1, CTLA-4, LAG-3, TIGIT, GITR, CXCR4, CD73, HER2, VEGF, CD20, CD40, CD11a, tissue factor (TF), PSCA, IL-8, EGFR, HER3, and HER4.

14. The method of any one of claims 1-13, wherein the cells are cultured at a single constant temperature over the whole production culture period.

15. The method of any one of claims 1-13, wherein the cells are cultured at a shifted temperature for some of culture period.
